(19) United States
(12) Patent Application Publication
(10) Pub. No.: US 2014/0323950 A1
(43) Pub. Date: Oct. 30, 2014

(54) SKIN LIGHTENER IN PHOTOTHERAPY

(71) Applicant: Merck Patent GmbH, Darmstadt (DE)

(72) Inventor: Corinna Wirth, Darmstadt (DE)

(21) Appl. No.: 14/354,156

(22) PCT Filed: Sep. 26, 2012
(86) PCT No.: PCT/EP2012/004019

§ 371 Date: Apr. 25, 2014

(30) Foreign Application Priority Data
Oct. 29, 2011 (DE) 10 2011 117 364.5

(51) Int. Cl. A61N 5/06

(52) U.S. Cl. CPC A61N 5/0621; A61N 5/0616; A61N 2005/0651; A61N 2005/0653
USPC 604/20; 424/62

(57) ABSTRACT

The invention relates, inter alia, to melanin synthesis inhibitors for use in treating skin illnesses, wherein the skin is additionally irradiated with an artificial radiation source. The invention further relates to cosmetic treatments.

The present invention relates, inter alia, to the use of melanin synthesis inhibitors for the treatment of skin diseases, where the skin is additionally to the radiation from an artificial radiation source. The present invention furthermore relates to cosmetic treatments, and to cosmetic and pharmaceutical preparations

Phototherapy (also called light therapy) is used in many therapeutic and cosmetic (aesthetic) areas. The areas of application here are very broad and include, for example, wound healing, the treatment of injuries, pain, the amelioration of side effects of chemotherapy, and radiotherapy.

Further typical areas of application are the therapeutic and/or cosmetic treatment of psoriasis, acne, atopic dermatitis, skin ageing, formation of skin wrinkles and cellulite.

It is known that skin is positively influenced in very different ways by irradiation with light. Thus, light can, for example, stimulate the metabolism of the mitochondria. It has been found that certain wavelengths stimulate cytochrome-C oxidase. The enzyme is responsible for the production of cellular energy in the form of ATP (adenosine triphosphate), which is involved in the energy transfer of biochemical processes and, as signal molecule, in the modulation of other biochemical molecules. After performance of phototherapy, cells exhibit increased metabolism, improved communication and become more resistant to stress.

Phototherapy is today carried out virtually exclusively on in-patients or out-patients in medical practices or hospitals under the direction of medically trained personnel. Furthermore, the irradiation sources are usually large and expensive equipment which does not allow mobile use or use outside the medical facilities, such as, for example, in the private living area or during daily work.

For the patients, this means that they must in all cases go to a medical facility in order to undergo phototherapy, which results in reduced acceptance of phototherapy amongst patients. In particular, cosmetic applications, which could easily be carried out by the user, checking themselves, but also many pharmaceutical or dermatological applications, also often fail for the reasons mentioned.

Furthermore, the costs for out-patient and in-patient treatment by means of classical phototherapy are very high, which results in an increase in the financial burden on the health systems.

It is therefore desirable to research and develop novel phototherapeutic treatment possibilities which result in greater acceptance amongst those affected, in high efficacy and in lower financial burdens.

Various approaches for introducing novel light sources for phototherapy have been described in the prior art. WO 98/46130 and US 6096066 describe arrays of LEDs (light-emitting diodes) for use in photodynamic therapy (PDT). LEDs are relatively small light sources, which, however, like conventional irradiation sources, represent point-form emitters. In addition, the production of the arrays is very complex and the device are not intended for mobile use.

GB 2360461 discloses a flexible fabric which is employed for POT. However, classical light sources are used here, where the light is propagated through optical fibres. The light sources are not suitable for use outside medical facilities. They are only suitable for use in medical facilities.

WO 93/21842 discloses a transportable device containing LEDs as light source for phototherapy. However, this device is intended for out-patient therapy and is not suitable for mobile and/or private use.

Rochester et al. disclose in GB 24082092 a flexible, medical light source which contains LEDs and is employed for diagnostic purposes in order to check blood values.

Vogle Klaus and Kallert Heiko disclose in EP 018180773 a device for the irradiation of the skin. As light source, use is made of OLEDs (organic light-emitting diodes). The device can be installed in pieces of clothing or plasters. OLEDs have various advantages over conventional light sources. Thus. OLEDs are not point emitters, but instead area emitters. Furthermore, OLEDs, owing to their structure, allow the production of a very thin light source for use in phototherapy.

Attili et al. (Br. J. Dermatol. 161(1), 170-173. 2009) disclosed a clinical pilot study in the course of which mobile light sources containing OLEDs were advantageously employed for the treatment of skin cancer.

The use of OLEDs in phototherapy has also been described by other authors (for example in EP 1444008). In addition, specific cosmetic, pharmaceutical and dermatological applications of OLEDs in phototherapy, such as, for example, the use of OLEDs for the treatment of psoriasis, acne, wrinkle ageing, inflammation, wound healing or jaundice (icterus), for example the treatment of jaundice of the newborn (US2010/0179469 A1), are prior art.

Further advantages of OLEDs over conventional light sources are their potential flexibility, and the possibility of being able to process them in large areas, for example by means of printing techniques, such as ink-jet or screen printing.

Besides the rather technical advantages that OLEDs have in phototherapeutic use, the psychological or emotional advantages of OLEDs and other radiation sources having similar properties have a further, very particular importance. Thus, as already described above, user acceptance of classical phototherapy is low in many areas of application. The disadvantage of classical methods can be overcome by the use of novel, thin irradiation sources, such as OLEDs. OLEDs and other radiation sources (such as, for example, OLECs) having similar properties can be built, for example, into plasters, bandages, pieces of clothing, headwear, sleeping bags or blankets, enabling phototherapy even in parallel with private or work activities and without significant restrictions.

Besides the advantageous technical effects, OLEDs also have positive psychological/emotional effects also in the phototerapeutic treatment of jaundice of the newborn. Jaundice of the newborn (icterus of the newborn or hyperbilirubinaemia of the newborn, lat. icterus neonatorum) describes the phenomenon that 60% of all mature healthy newborn develop more or less pronounced jaundice (icterus) in the first days of life. The jaundice arises through increased incorporation of bilirubin, a degradation product of the red blood dye haemoglobin, into the skin. The incorporation generally achieves its maximum on about the fifth day of life and then gradually reduces. This is a natural process and usually completely harmless.

In some cases, jaundice of the newborn is not a medical problem, since it is basically normal or physiological as a frequent phenomenon. In many other cases, however, the amount of fat-soluble bilirubin exceeds a threshold value, so that it can cross the blood-brain barrier and pass from the bloodstream into the brain, where it deposits in certain structures. The brain can be permanently damaged by this, so-called kernicterus occurs. This complication is a particular risk for children whose blood-brain barrier does not function or does not yet function completely for other reasons. These include all premature babies, but also full-term babies with acute diseases which result in impairment of the blood-brain barrier. These include, for example, severe lack of oxygen during birth (asphyxia), overacidification of the body caused by this or by other causes (acidosis), hypoglycaemia, albumin deficiency (hypalbuminaemia), shock or bacterial infections (sepsis).

Furthermore, it may be that the liver of newborn, in particular the liver of premature babies, does not yet produce a special enzyme, or only does so in inadequate amounts. This enzyme is so-called glucuronyl transferase, which is involved in the conversion of fat-soluble bilirubin into a water-soluble product. A deficiency of the enzyme results in it being impossible for the fat-soluble bilirubin to be excreted via the bile or kidney and in it consequently accumulating further in the body.

In the case of all newborn whose jaundice exceed age-related threshold values set by the expert societies, treatment by irradiation with blue light of wavelength 460 nm must be commenced in good time in order to prevent kernicterus. The irradiation of the skin converts the fat-soluble bilirubin into a water-soluble product, which can be excreted via the bile and via the kidneys.

For the treatment of jaundice in newborn, the babies are typically irradiated under classical blue-light lamps with light of a wavelength of 460 nm. The irradiation of newborn is frequently carried out here inside a baby incubator, but in any case without close body contact with the parents. The irradiation is usually carried out in a number of units per day over a period of 2 to 14 days. In order to avoid damage to the retina, the babies are blindfolded during this. In practice, the therapy therefore repeatedly results in an emotional stress situation for child and parents. The use of OLEDs and similar irradiation sources in phototherapy enables at least some of the disadvantages of the classical treatment of babies to be overcome. Thus, the novel light sources can be used, for example, in blankets or sleeping bags, enabling at least direct body contact between child and parents to be established during the irradiation.

However, side effects also occur with phototherapy. Thus, for example, the retina of the eye can be damaged by the high-energy light rays. In addition, increased water and salt losses arise via the skin, and dried-out skin and inflammation of the conjunctiva of the eyes (conjunctivitis) and of the skin (dermatitis) and disorders of heat regulation or other permanent skin damage may occur. There is therefore a need to research novel possibilities which result in a reduction in the said disadvantages while retaining the effect of phototherapy. In principle, two variants are conceivable here. On the one hand, it is conceivable to increase the efficacy of photo-therapy with an unchanged radiation intensity and wavelength, so that shorter irradiation times are necessary, which should result in fewer side effects. On the other hand, it is also conceivable to modify radiation parameters, for example the radiation intensity, so that the irradiation is less critical for the skin or the tissue to be irradiated, where the cosmetic and/or pharmaceutical and/or dermatological effect should be retained.

The advantage may of course also lie in a combination of the two effects. It is thus conceivable to develop a phototherapy in which the irradiation times are shortened, the efficacy can be increased and in addition side effects can be reduced.

The present invention is based on the object of overcoming the disadvantages indicated in the prior art and developing novel, effective possibilities.

Surprisingly, it has been found that the disadvantages described can be considerably reduced by the use of skin-lightening compounds in photo-therapy. Skin-lightening compounds reduce the concentration of melanin in the skin in various ways, making the skin more transmissive to radiation.

In this case, either the radiation intensity can be reduced during photo-therapy in order to obtain the desired effect or the irradiation time can be shortened at the same radiation intensity. However, it is also possible to reduce both the radiation intensity and the treatment duration in order to obtain the desired therapeutic effect. In each case, this results in a reduction in the side effects by the radiation used, with the therapeutic effect remaining unchanged. Precise setting of the parameters, such as treatment duration and radiation intensity, is readily possible for the person skilled in the art by means of routine experiments. The principle can be applied to all phototherapeutic applications in order to be able to exploit the therapeutic benefit of light better and/or to reduce the side effects of radiation.

The present invention relates to skin-lightening compounds for the therapeutic and/or cosmetic treatment of the skin with simultaneous use of phototherapy, where an artificial radiation source is preferably used for the phototherapy.

The meaning of the term “skin-lightening compound” is well known to the person skilled in the art, and it is readily possible for him to fall back on a multiplicity of alternative skin-lightening compounds from the prior art which have the same effect without being inventive at the same time. A very large number of compounds which have a skin-lightening property are described in the prior art.

A feature that the skin-lightening compounds have in common is that they reduce the concentration of melanin in the skin. Melanins are reddish, brown or black pigments which are formed by the enzymatic oxidation of tyrosine and which cause colouring of the skin, hair or eyes in humans. They furthermore also occur, for example, in other vertebrates, micro-organics and plants. Melanin is formed in vertebrates in the melanocytes of the skin and in the retina of the eye. The formation of melanins in the skin results in natural light protection.

The biological or biochemical mechanisms which form the basis of the skin-lightening action of the compounds can in some cases be very different. S. Briganti et al. (Pigment Cell Res. 16: 101-110, 2003) disclose three basic, important concepts for depigmentation of the skin:
(i) Regulation of the transcription and/or activity of tyrosinase, 5,6-dihydroxyindole-2-carboxylic acid oxidase (TRP-1—tyrosine-related protein-1). L-dopachrome tautomerase (TRP-2—tyrosine-related protein-2) and/or peroxidase.
(ii) Uptake and distribution of melanosomes in keratinocytes which are ready for reception.
(iii) Degradation of melanin and melanosomes and conversion of pigmented keratinocytes.

In this respect, skin-lightening compounds are typically summarised in different categories, namely those which cause a reduction in the melanin concentration in the skin before melanin synthesis (for example; regulation of tyrosinase transcription by C₂-ceramide or tretinoin; regulation of tyrosinase glycosylation by calcium D-pantetheine S-sulfonate), those which cause a reduction in the melanin concentration in the skin during melanin synthesis (for example: inhibition of tyrosinase by hydroquinone, kojic acid, 4-hydroxyanisole, methyl gentisate, 4-S-cystaminylphenol and derivatives thereof, ellagic acid, arbutin, resveratrol, aloesin, oxyresveratrol and azelaic acid; inhibition of peroxidase by methimazole phenols/catechols; product reduction and free-radical scavenger for reactive oxygen species (ROS) by ascorbic acid. alpha-tocopherol, ascorbyl palmitate. D and L alpha-tocopherol ferulate, magnesium leascorbyl 2-phosphate, hydrocoumarins) and those which cause a reduction in the melanin concentration in the skin after melanin synthesis (for example: degradation of tyrosinase by linoleic acid and alpha-linoleic acid; inhibition of the transfer of melanosomes by serine protease inhibitors, niacinamides, lectins and neoglycoproteins, soya bean/milk extract; increase in the conversion of the skin by lactic acid, retinoic acid, glycolic acid, linoleic acid, liquiritin).

Further common skin-lightening compounds which are well known to the person skilled in the art are listed below by way of example: pantothenic acid and derivatives or salts thereof (sodium or calcium pantothenate, pantetheines; pantethine ((2R)-2,4-dihydroxy-3,3-dimethyl-N-[2-(2-sul-fanylethylcarbamoyl)ethyl]butanamide); phosphopantetheine; etc.), hydroquinone or derivatives thereof (hydroquinone glucosides, such as arbutin etc.), glucosamines or derivatives thereof (glucosamine esters, such as acetylglucosamine, glucosamine ethers, such as glucosamine methyl ether; etc.), mercaptoamines, hinokitiol or derivatives thereof (hinokitiol glucoside etc.), azelaic acid and salts and derivatives thereof (monoesters of azelaic acid, such as azelaic acid monoalkyl esters; diesters of azelaic acid, such as azelaic acid dialkyl esters etc.), tocopherols (alpha-toco-pherol, beta-tocopherol, gamma-tocopherol, delta-tocopherol, etc.), ubiquinones (coenzyme Q₆ (CoQ₆), coenzyme Q₇ (CoQ₇), coenzyme Q₈ (CoQ₈), coenzyme Q₉ (CoQ₉), coenzyme Q₁₀ (CoQ₁₀), etc.), carotenes (carotene, lutein, violaxanthine, spirilloxanthine, spheroidene, etc.), flavones (flavone, apigenin, luteolin and glucosides thereof etc.), isoflavones or derivatives thereof (isoflavone, isoflavone glucoside etc.), flavanones or derivatives thereof (naringenin, eriodictyol, naringin, etc.), catechines (catechine, catechine gallate, gallocatechine, etc.), flavonols (kaempferol, quercetin, myricetin and glucosides thereof etc.), glycyrrhizin and derivatives and salts thereof (dipotassium or monoammonium salts of glycyrrhizin etc.), glycyrrhetinic acid and derivatives and salts thereof (glycyrrhetinic acid alkyl esters, such as stearyl glycyrrhetinate etc.), glabridin, kojic acid and derivatives or salts thereof (kojic acid monoalkyl esters, such as kojic acid monobutyrate, kojic acid monocaprylate and kojic acid monostearate, kojic acid monopalmitate or a difatty acid ester of kojic acid selected from kojic acid dipalmitate, kojic acid dibutyrate, kojic acid dioleate and kojic acid distearate or kojic acid monocinnamoate or kojic acid monobenzoate, rucinol, ellagic acid and derivatives or salts thereof (ellagic acid ethers, such as ellagic acid tetramethyl ether; acyl derivatives of ellagic acid, such as ellagic acid tetraacetate and ellagic acid tetra-benzoate etc.), niacinamides, 4-hydroxyanisole, 4-tert-butylphenol, gentisinic acid, aloesin, resveratrol, oxyresveratrol, 6-hydroxy-3,4-dihydro-coumarin and derivatives thereof, 4-S-cystaminylphenol, glutathione and derivatives or salts thereof (glutathione, S-acylglutathiones, such as S-lactoylglutathione; N,S-diacylglutathione diesters, such as N,S-dioctanoylglutathione distearyl and N,S-dipalmitoylglutathione dicetyl; etc.), resorcinol or derivatives thereof (resorcinol; alkylated resorcinol, such as 4-n-butylresorcinol, 4-isoamylresorcinol, 4-cyclohexylresorcinol and 5-methylresorcinol; halogenated resorcinol, such as 4-chlororesorcinol and 4-bromoresorcinol.

Skin-lightening compounds in the sense of the present invention are not taken to mean compounds which are typically employed as light-protection filters and/or UV filters.

Still further common skin-lightening compounds which are well known to the person skilled in the art are the following plant extracts: Job's tears (Coix Lacryma-jobi), hamamelis (Hamamelis mamelodis), strawberry saxifrage (Saxifraga stolonifera), agarwood (Aquilaria agallocha), tea (Thea sinensis), Japanese knotweed (for example Polygonum cuspidatum), lemon balm (Melisa officinal's), thyme (Thymus vulgaris), mugwort (for example Artemisia capillaris), yarrow (Achillea milefolium), Canadian St. John's wort (for example Hypericum erectum), St. John's wort (Hypericum perforatum), peony (for example Paeonia actiflora), liquorice (Glycyrrhiza glabra), Chinese liquorice (Glycyrrhiza uralensis), Mitracarpus scaber extract, bearberry (Arctostaphylos uva-ursi), mulberry (Morus bombycis), white mulberry (Morus alba), paper mulberry (Broussonetia papyrifera), Surinam cherry (Eugenina uniflora), Indian gooseberry (Emblica officinalis), sophora (Sophora flavescens), okamura (Dictyopteris prolifera), algae (for example Dictyota linearis, Sargassum fusiforme, Lomentaria catenata, Rhodymenia palmata, Sargassum ringgoldianum, coreanum, Sargassum confusum, Sargassum yezoense, Sphaerotrichia divaricata), clubmoss mountain heather (Cassiope lycopodioides), sundew (Drosera rotundifolia), sundew (Drosera spatulata), lavender/Lavandula officinalis), gold-thread (Coptis japonicus) Japanese ground laurel (Epigaea asiatica), mayflower (Epigaea repens), passionflower (for example Passiflora incarnata), passionfruit (Passiflora edulis), passionflower (for example Passiflora caerulea), passionflower (Passiflora altebilobata), passion-flower (Passiflora moluccana), wild pansy (Viola tricolor), sweet violet (Viola odorata), clematis (Clematis florida), clematis (Clematis patens), gutta-percha tree (Eucommia ulmoides), spindle bush (Euonymus trichocarpus Hayata), spindle bush (Euonymus oxyphyllus), asparagus (Asparagus officinalis), pea (Pisum sativum), rose (Rosa multiflora), Chinese skull-cap (Scutellaria baicalensis), rasperry (Rubus idaeus), blackberry (Rubus rubus), evergreen wisteria (Millettia reticulata), Acanthopanax bark (Acanthopanax gracilistylus), hawthorn (Crataegus cuneata), senna (Cassia senna), soya (Glycine max), angelica (Angelica acutilob), grape (Vitex viniflora), aloe (Aloe ferox), marshmallow (Althaea officinalis), arnica (Arnica montana), stinging nettle (Urtica dioica), cork tree (Phellodendron amurense), chamomile (Matricaria chamomilla), honeysuckle (Lonicera japonica), watercress (Nasturtium officinale), comfrey (Symphytum officinale), sage (Salvia officinale), birch (Betula platyphylla), marigold (Calendula officinalis), Japanese red elder (Sambucus sieboldiana), bulrush (Typha latifolia), soap nut tree (Sapindus mukurossi), vetch (Astragalus sinicus) eucalyptus (Eucalyptus officinalis), ginkgo (Ginkgo biloba), wolfberry (Lycium chinense), micromelum (Micromelum pubescens), melothria (Melothria indica), Mangifera indica, firethorn (Pyracantha fortuneana).

A further group of compounds having skin-lightening properties is disclosed in WO 2007/121845. Compounds of the formula (1) or (2) or (3) are particularly preferred from these.

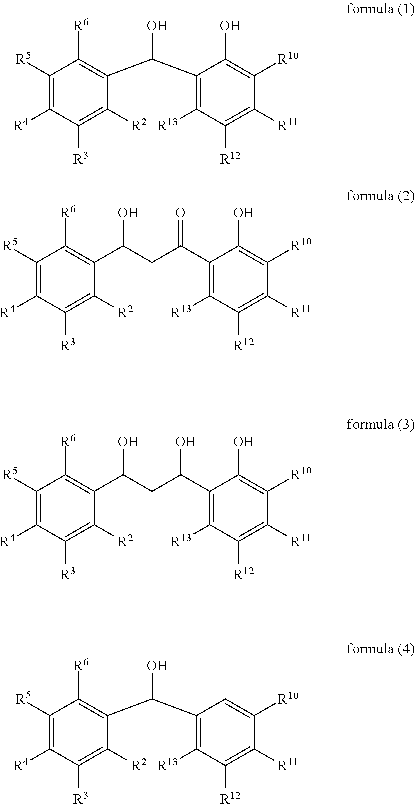

where

R² to R⁶ and R⁹ to R¹³ are each selected, independently of one another, from H, OH, straight-chain or branched C₁- to C₂₀-alkoxy groups, where the alkyl chains may each also be interrupted by oxygen or nitrogen, straight-chain or branched C₁- to C₂₀-alkyl groups, where the alkyl chains may each also be interrupted by oxygen or nitrogen,

straight-chain or branched C₃- to C₂₀-alkenyl groups,

straight-chain or branched C₁- to C₂₀-hydroxyalkyl groups, where the hydroxyl group may be bonded to a primary or secondary carbon atom of the chain and furthermore the alkyl chains may each also be interrupted by oxygen or nitrogen,

straight-chain or branched C₁- to C₂₀-hydroxyalkoxy groups, where the hydroxyl group(s) may be bonded to primary or secondary carbon atoms of the chain and furthermore the alkyl chain may also be interrupted by oxygen,

straight-chain or branched C₁- to C₂₀-alkylamino groups,

straight-chain or branched C₁- to C₂₀-dialkylamino groups,

or R² to R⁶ and R⁹ to R¹³ each stand, independently of one another, for a carboxylic acid, phosphoric acid, sulfonic acid, sulfuric acid or sulfone function, which may optionally be esterified or alkylated with straight-chain or branched C₁- to C₂₀-alkyl groups or straight-chain or branched C₃- to C₂₀-alkenyl groups,

or are salts of the compounds of the formula (1) to (4).

For the purposes of the present invention, the following compounds from the laid-open specification WO 2007/121845 having the formulae (5) to (10) are to be regarded as furthermore preferred compounds having skin-lightening properties.

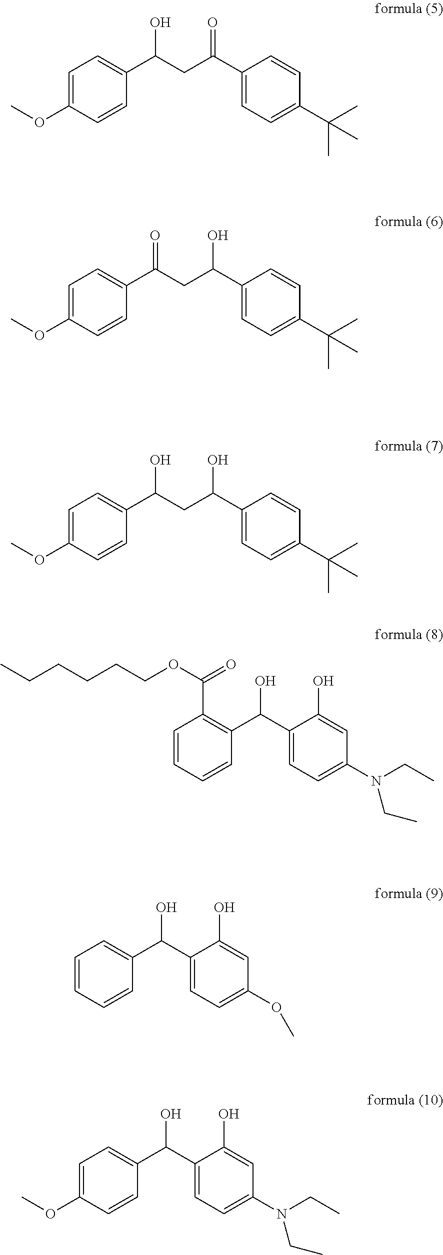

Skin in the sense of the present invention is taken to mean all parts of the animal and human body which produce melanin for protection against radiation

Furthermore, skin in the sense of the present invention is taken to mean both animal and also human skin, preferably human skin.

In principle, the use according to the invention of skin-lightening compounds in phototherapy can be employed for any pharmaceutical and/or dermatological and/or cosmetic therapy or use. In any case, the effect of the radiation used can be increased and/or the side effects of damaging radiation can be reduced. Typical areas of application are, without being limiting, psoriasis, atopic dermatitis, eczema of the skin, inflammation of the skin, acne, the reduction and/or prevention of skin wrinkle formation, skin ageing, pigment defects (for example freckles, age spots), reddening of the skin, blackheads, and cellulite, stimulation of hair growth and the treatment of jaundice, in particular in newborn,

The advantages of the present invention are apparent, in particular, in the case of skin which exhibit a certain tanning effect, This tanning effect can be categorised by means of a classification which is established amongst dermatologists. This is the so-called Fitzpatrick scale, according to which the skin is classified in different types I to VI.

Besides natural radiation, the phototherapy itself can also result in tanning of the irradiated skin areas, depending on the wavelength employed, which reduces the effect of the phototherapy. This effect of the reduction in the efficacy of the phototherapy occurs in all skin types I to VI. This effect is particularly pronounced in the case of skin types III and higher. Consequently, it is desirable to reduce the effect of tanning, which is possible in accordance with the invention through the use of skin-lightening compounds. In this respect, the present invention relates in a preferred embodiment to skin-lightening compounds for the therapeutic and/or cosmetic treatment of skin of type III to VI, very preferably skin of type IV to VI and very particularly preferably skin of type V and VI, with simultaneous use of phototherapy, where an artificial radiation source is preferably used for the phototherapy.

In a further embodiment of the present invention, the tanning effect by natural irradiation and/or by the phototherapy itself can also be reduced further by the additional use of light-protection filters, which is described in greater detail below.

The present invention preferably relates to skin-lightening compounds for the therapeutic and/or cosmetic treatment of the skin with simultaneous use of phototherapy by means of an artificial radiation source, where the aim is to treat jaundice, preferably jaundice of the newborn.

In newborn, melanin synthesis commences within a few days and is initiated by visible light. There are reports that negroid and Asiatic newborn tan during phototherapy against jaundice. This emanates purely from visible light, since light sources for the treatment of jaundice emit very little UV. Since melanin absorbs at the efficient wavelength around 460-470 nm, the formation is counterproductive to success of the treatment.

Preference is therefore given in the sense of the present invention to skin-lightening compounds for the therapeutic and/or cosmetic treatment of the skin with simultaneous use of phototherapy by means of an artificial radiation source, where jaundice of negroid and Asiatic newborn is treated. Negroid and Asiatic newborn in the context of the present invention are taken to mean children who have a skin type III to VI, preferably a skin type IV to VI and very particularly preferably a skin type V and VI.

In a further particularly preferred embodiment, one or more light-protection factors can be employed in addition to the use of skin-lightening compounds.

The present invention furthermore preferably relates to compound according to claim 1, characterised in that the compound are employed in the course of a cosmetic treatment for the elimination and/or reduction and/or prevention of skin ageing, blackheads, acne, formation of skin wrinkles and cellulite, of pigment defects (for example freckles and age spots), and/or for the stimulation of hair growth, is preferably employed for the elimination and/or reduction and/or prevention of skin wrinkles.

The artificial radiation source which can be used in accordance with the invention is in principle any radiation source which is used in photo-therapy. This can thus be classical lamps (for example UV or IR lamps), lasers or light-emitting diodes. Particularly advantageous for the purposes of the present invention are radiation sources having very small dimensions. These include light-emitting diodes (LED); organic light-emitting diodes (OLED), polymeric light-emitting diodes (PLED) and organic lightemitting electrochemical cells (OLEG. LEC or also LEEC), preferably OLEDs, PLEDs and OLECs and very preferably OLEDs and PLEDs.

The structure and production of organic electroluminescent devices (for example OLEDs, PLEDs, OLECs) in which organic semiconductors are employed as functional materials is well known to the person skilled in the art from the prior art and is described, for example, in U.S. Pat. No. 4,539,507, U.S. Pat. No. 5,151,629, EP 0676461 and WO 98/27136.

OLEDs and PLEDs (polymeric light-emitting diodes) have a typical layer structure, where the number of layers in an OLED comprising small organic molecules is usually lower than the number of layers in a PLED. The organic functional materials used in an OLED or PLED can be all materials considered by a person skilled in the art in the area. OLEDs/PLEDs comprise, for example, organic semiconductors, organic metal complexes, hole-blocking materials (HBM), hole-transport materials (HTM), hole-injection materials (HIM), electron-blocking materials (EBM), electron-transport materials (ETM), electron-injection materials (EIM), exciton-blocking materials (ExBM), host materials, matrix materials, emitters, fluorescent emitters, phosphorescent emitters and dyes. PLEDs and/or OLEDs furthermore comprise electrodes (anode, cathode). Furthermore, OLEDs and PLEDs may comprise further materials, such as, for example, buffer materials and encapsulation materials. Typical structures of OLEDs and PLEDs and common materials for electroluminescent devices are disclosed, for example, in WO 2004/058911 and in WO 2008/011953.

The structure of OLECs (organic light-emitting electrochemical cells, also called LEC or LEEC) and materials used in OLECs are well known to the person skilled in the art. Besides the materials used in OLEDs/PLEDs, ionic materials are employed in OLECs, since, in contrast to OLEDs/PLEDs, the charge transport in OLECs takes place through mobile ions. Materials such as ionic transition-metal complexes (iTMCs) and ionic liquids are therefore employed in OLECs. More details in this respect have been published, for example, by Pei et al. in Science, 1995, 269, 1086.

In general, the OLED/PLED/OLEC light sources have a planar or a more or less planar layer structure. However, the light sources may also be in fibre form. Organic light-emitting fibres have been disclosed, for example, in U.S. Pat. No. 6,538,375 B1, US 2003/0099858 and in Brenndan O′Connor et al. (Adv. Mater. 2007, 19, 3897-3900).

With the aid of the fibres, flexible, plastic and elastic devices for the treatment of skin areas of any surface nature can be produced. The emitted wavelength can be adjusted without difficulties by the person skilled in the art through the use of different emitters. The radiation sources thus enable homogeneous irradiation of the skin.

An example of the typical structure of an OLED is given below: optionally a first substrate,
an anode,
optionally a hole-injection layer (HIL),
optionally a hole-transport layer (HTL) and/or an electron-blocking layer (EBL),
an emission layer (EML)
optionally an electron-transport layer (ETL) and/or hole-blocking layer (HBL),
optionally an electron-injection layer (EIL)
a cathode,
optionally a second substrate.

In addition to the said layers, OLEDs may comprise further layers, such as, for example, an exciton-blocking layer between the emission layer and an electrode.

A PLED is an OLED comprising one or more polymers in the emission layer. The polymers are typically processed from solution. A possible layer sequence for PLEDs is the following: anode/HIL or buffer layer/interlayer/EML/cathode. The interlayer has both hole-transport and electron-blocking properties. Further details on interlayers in PLEDs are disclosed in WO 2004/084260 A2.

Organic light-emitting electrochemical cells (OLECs) comprise two electrodes and a mixture of an electrolyte and photoluminescent species between the electrodes (Pei et al., Science 1995, 269, 1086). OLECs and OLEDs have certain similarities with respect to their layer structure. However, OLECs have advantages with respect to the selection of potential materials for the electrodes. Furthermore, the layers in OLECs may have a greater thickness. Overall, OLECs are simpler to produce than OLEDs.

The said light sources may comprise any further material which the person skilled in the art would consider in this respect. These materials can be colour converters or coloured filters. The devices may comprise down-conversion materials.

The colour converters can be organic or inorganic materials. Colour converters can be selected from the group of the phosphor materials used in fluorescent displays or lamps or CRTs (cathode-ray tubes).

Further radiation sources can be added easily and without being inventive by the person skilled in the art for the purpose indicated.

The light source itself may either continuously emit light or radiation of a specific wavelength or of a specific wavelength range or light of a specific pulse sequence. The person skilled in the art will be able to adapt the pulse sequence depending on the therapeutic or cosmetic application.

Particularly preferred skin-lightening compound in the sense of the present invention are selected from the group consisting of hydroquinone (1,4-dihydroxybenzene), kojic acid, arbutin, aloesin, niacinamide, vitamin C and derivatives of vitamin C, rucinol, plant extracts from Morus alba, Phyllanthus emblica or Eugenia uniflora.

The radiation emitted by the radiation source can have any cosmetically and/or therapeutically appropriate wavelength or wavelength range. A preferred wavelength and/or a preferred wavelength range in the sense of the present invention is in the range between 250 and 2000 nm, furthermore preferably between 270 and 1800 nm, very preferably between 290 and 1600 nm, very particularly preferably between 300 and 900 nm and especially preferably between 400 and 550 nm.

The wavelength or wavelength range can be selected in each case application-specifically by the person skilled in the art without inventive step. To this end, assignments between wavelengths and phototherapeutic applications are already well known in the prior art. Some of these known assignments are shown below, without being restrictive. They furthermore represent preferred wavelengths or ranges of wavelengths which can be emitted by the radiation source corresponding to the present invention: psoriasis—290 to 330 nm, in particular 311 nm; atopic eczema—300 to 340 nm, in particular 320 nm; inflammation—380 to 450 nm, in particular 405 nm and 420 nm; acne—390 to 440 nm, in particular 415 nm; atopic eczema—400 to 500 nm; seasonal affective disorder (SAD)—440 to 500 nm, in particular 460 nm and/480 nm; jaundice—450 to 480 nm; blackheads—480 to 520 nm, in particular 500 nm; acne—500 to 750 nm; dermatitis—540 to 610 nm, in particular 560 nm and 590 nm; inflammation—540 to 610 nm; reduction of skin reddening—560 nm and higher; anti-skin wrinkle formation—570 to 610 nm, in particular 590 nm; acne—640 to 680 nm, in particular 660 nm; wound healing—640 to 900 nm, in particular 660 nm, 720 nm and 880 nm; oedema—830 to 870 nm, in particular 850 nm.

Particular preference is given here to the wavelengths for acne, skin wrinkling and jaundice, very particularly preferably those for jaundice.

The present invention furthermore relates to the combination of at least one skin-lightening compound and at least one compound which has light-protection properties, for example UVNIS/IR filters, for the therapeutic and/or cosmetic treatment of the skin with simultaneous use of photo-therapy, where an artificial radiation source is preferably used for the phototherapy. In the case of the use of additional light-protection filters, it should be noted when making a suitable selection thereof that although they should absorb radiation, they should not absorb the phototherapeutic radiation. The additional use of substances having light-protection properties has the effect that the action of the phototherapy according to the invention can be increased further. The compounds having light-protection properties prevent or reduce tanning of the skin and consequently make the skin more transmissive to the phototherapeutic radiation.

The present invention furthermore relates to pharmaceutical composition comprising at least one skin-lightening compound and optionally at least one further pharmaceutical assistant.

The pharmaceutical composition according to the invention preferably comprises 4, very preferably 3, very particularly preferably 2 and especially preferably precisely one skin-lightening compound and optionally at least one further pharmaceutical assistant.

All assistants typically employed can be used here. Some of them are indicated below.

Furthermore, the present invention relates to pharmaceutical compositions which, besides the at least one skin-lightening compound and besides the optional at least one further pharmaceutical assistant, also comprises at least one further active compound.

It is preferred here for the composition to comprise precisely one skin-lightening compound and 3, very preferably 2 and very particularly preferably precisely one further active compound.

A “drug”, “medicament” and a “pharmaceutical composition”, “pharmaceutical formulation” or “pharmaceutical preparation” here is any composition which can be employed in the prophylaxis, therapy, progress control or aftertreatment of patients who, at least temporarily, exhibit a pathogenic modification of the overall condition or the condition of individual parts of the patient organism, preferably of disorders of the skin

The present invention furthermore relates to cosmetic composition comprising at least one skin-lightening compound and optionally at least one further cosmetic assistant.

The cosmetic composition according to the invention preferably comprises 4, very preferably 3, very particularly preferably 2 and especially preferably precisely one skin-lightening compound and optionally at least one further cosmetic assistant.

The present invention furthermore relates to cosmetic compositions which, besides the at least one skin-lightening compound and besides the optional at least one further cosmetic assistant, also comprises at least one further active compound.

It is preferred here for the composition to comprise precisely one skin-lightening compound and 3, very preferably 2 and very particularly preferably precisely one further active compound. The cosmetic compositions may also comprise at least one further active compound

The further active compound is preferably used in the same composition in which the skin-lightening compound is also present. However, it is also possible for the further active compound and the skin-lightening compound to be present in separate administration forms. Thus, for example, it is conceivable for the further active compound to be administered orally, whereas the skin-lightening compound is applied topically by means of a cream, ointment, lotion, emulsion, etc.

The further active compound for the cosmetic or pharmaceutical compositions is preferably selected from the group of antioxidants, vitamins, UV filters, light filters (VIS, IR), anti-inflammatory agents, antimicrobial active compounds, skin-moisturising active compounds, ageing-inhibiting active compounds (anti-ageing active compounds) and anticellulite active compounds. Preference is given to the use in combination with at least one or more UV filters.

The further active compound is very particularly preferably selected from the group of the active compounds employed dermatologically. These include, in particular, the corticoids, glucocorticoids (for example cortisone, corticosterone and cortisol), mineral corticoids (for example aldosterone and desoxycorticosterone), synthetic corticoids (for example prednisone and prednisolone, methylprednisolone, triamcinolone, dexamethasone, betamethasone and paramethasone).

Furthermore, photodynamic active compounds can be used as further active compounds. These include, for example, porphyrins, chlorophylls, and dyes, aminolevulinic acid, methyl aminolevulinate, levulinic acid, silicone phthalocyanine, m-tetrahydroxyphenylchlorine , and mono-L-aspartyl chlorine, Photofrin® sodium porfirmer, Visudyne® (verteporfin), Foscan (temoporfin), Metvix ®(methyl (5-amino-4-oxopentanoate)), Cysview™ (hexaminolevulinic acid), Laserphyrin® (talaporfin), Photochlor (2-(1-hexyloxyethyl)-2-devinyl pyropheophorbide-a (HPPH)),Photrex ® (rosta-porfin), psoralen (for example 8-methoxy-psoralen).

The present invention furthermore preferably relates to a pharmaceutical composition comprising a skin-lightening compound and one or more UV filters.

For the purposes of the present invention, the term “agent”, “preparation” or “formulation” is also used synonymously alongside the term “composition”.

The compositions of the present invention are usually compositions which can be applied topically, such as, for example, cosmetic or pharmaceutical or dermatological formulations or medicinal products. Can be applied topically in the sense of the invention means that the preparation is applied externally and locally, i.e. that the preparation must be suitable, for example, for being able to be applied to the skin. In this case, the preparations comprise a cosmetically, pharmaceutically or dermatologically suitable vehicle and, depending on the desired property profile, optionally further suitable ingredients.

The compositions may include or comprise, essentially consist of or consist of the necessary or optional constituents mentioned above and/or below. All compounds or components which can be used in the preparations are either known and commercially available or can be synthesised by known processes.

The pharmaceutical, cosmetic and dermatological compositions according to the invention preferably comprise 0.01 to 99% by weight of the skin-lightening compound, based on the total weight of the composition. An amount of 0.05 to 30% by weight is preferably employed, particularly preferably 0.1 to 10% by weight. The person skilled in the art is presented with absolutely no difficulties in appropriately selecting the amounts depending on the intended effect of the composition.

In principle, all UV filters can be employed in the pharmaceutical, cosmetic and dermatological compositions according to the invention. Particular preference is given to UV filters whose physiological acceptability has already been demonstrated. There are many proven substances known from the specialist literature both for UVA and also UVB filters. The compounds shown in the following lists should only be regarded as examples. Other UV filters can of course also be used.

Preferred preparations may comprise organic UV filters, so-called hydrophilic or lipophilic sun-protection filters, which are effective in the UVA region and/or UVB region and/or IR and/or VIS region (absorbers). These substances can be selected, in particular, from p-aminobenzoic acid derivatives, salicylic acid derivatives, β,β-diphenylacrylate derivatives, camphor derivatives, triazine derivatives, cinnamic acid derivatives and polymeric filters and silicone filters, which are described in the application WO 93/04665. Further examples of organic filters are indicated in the patent application EP-A 0 487 404. The said UV filters are usually named below in accordance with INCI nomenclature.

Particularly suitable for a combination are: para-Aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed by BASF under the name “Uvinul P25”.

Salicylates: Homosalate marketed by Merck under the name “Eusolex HMS”; Ethylhexyl salicylate, for example marketed by Symrise under the name “Neo Heliopan OS”; Dipropylene glycol salicylate, for example marketed by Scher under the name “Dipsal”; TEA salicylate, for example marketed by Symrise under the name “Neo Heliopan TS”.

β,β-Diphenylacrylate derivatives: Octocrylene, for example marketed by Merck under the name “Eusolex® OCR”; “Uvinul N539” from BASF; Etocrylene, for example marketed by BASF under the name “Uvinul N35”.

Benzophenone derivatives: benzophenone-1, for example marketed under the name “Uvinul 400”; benzophenone-2, for example marketed under the name “Uvinul D50”; benzophenone-3 or oxybenzone, for example marketed under the name “Uvinul M40”; benzophenone-4, for example marketed under the name “Uvinul MS40”; benzophenone-9, for example marketed by BASF under the name “Uvinul DS-49”; benzophenone-5, benzophenone-6, for example marketed by Norquay under the name “Helisorb 11”; benzophenone-8, for example marketed by American Cyanamid under the name “Spectra-Sorb UV-24”; benzophenone-12 n-hexyl 2-(4-diethylamino-2-hydroxybenzoyl) benzoate or 2-hydroxy-4-methoxybenzophenone, marketed by Merck, Darmstadt, under the name Eusolex® 4360.

Benzylidenecamphor derivatives: 3-benzylidenecamphor, for example marketed by Chimex under the name “Mexoryl SD”; 4-methylbenzylidene-camphor, for example marketed by Merck under the name “Eusolex 6300”; benzylidenecamphorsulfonic acid, for example marketed by Chimex under the name “Mexoryl SL”, Camphor benzalkonium methosulfate, for example marketed by Chimex under the name “Mexoryl SO”; terephthalylidene-dicamphorsulfonic acid, for example marketed by Chimex under the name “Mexoryl SX”; polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”; disodium phenyl dibenzimidazole tetrasulfonate, for example marketed by Symrise under the name “Neo Heliopan AP”.

Phenylbenzotriazole derivatives: Drometrizol trisiloxane, for example marketed by Rhodia Chimie under the name “Silatrizole”; Methylenebis(benzo-triazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: Ethylhexyltriazone, for example marketed by BASF under the name “Uvinul T150”; Diethylhexylbutamidotriazone, for example marketed by Sigma 3V under the name “Uvasorb HEB”; 2,4,6-tris(diiso-butyl 4′-aminobenzalmalonate)s-triazine or 2,4,6-Tris(biphenyl)-1,3,5-triazine marketed by BASF as Tinosorb A2B; 2,2′46-(4-methoxyphenyl)-1,3,5-triazine-2,4-diyl]bis[5-(2-ethylhexyl)oxy]phenol; marketed by BASF as Tinosorb S; N2,N4-bis[4-[5-(1,1-dimethylpropyl)-2-benzoxazolyl]-phenyl]-N6-(2-ethylhexyl)-1,3,5-triazine-2,4,6-triamine marketed as Uvasorb K 2A by Sigma 3V.

Anthraniline derivatives: Menthyl anthranilate, for example marketed by Symrise under the name “Neo Heliopan MA”.

Imidazole derivatives: ethylhexyldimethoxybenzylidenedioxoimidazoline propionate.

Benzalmalonate derivatives: polyorganosiloxanes containing functional benzalmalonate groups, such as, for example, Polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl)imino]-6-(2-ethylhexyl)imino-1,3,5-triazine, for example marketed by Sigma 3V under the name Uvasorb K2A, and mixtures comprising this.

Piperazine derivatives, such as, for example, the compound

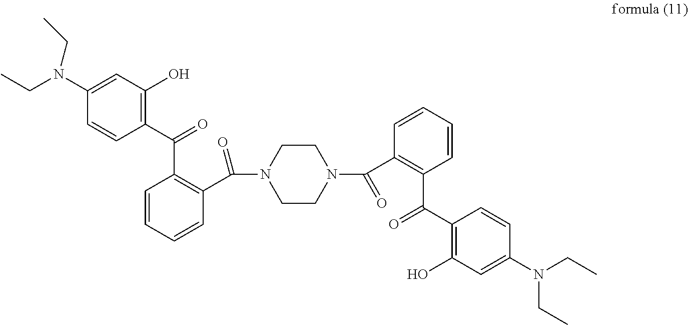 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

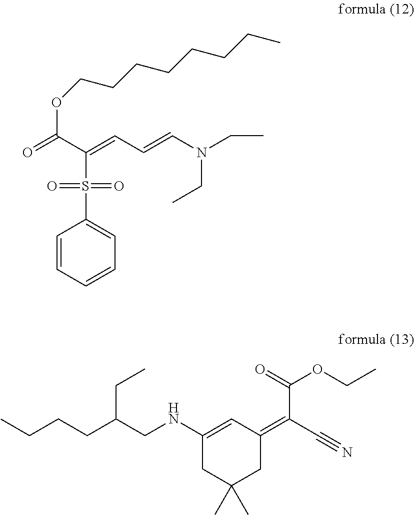

or.

It is also possible to use UV filters based on polysiloxane copolymers having a random distribution in accordance with the following formula, where, for example, a=1.2; b=58 and c=2.8:

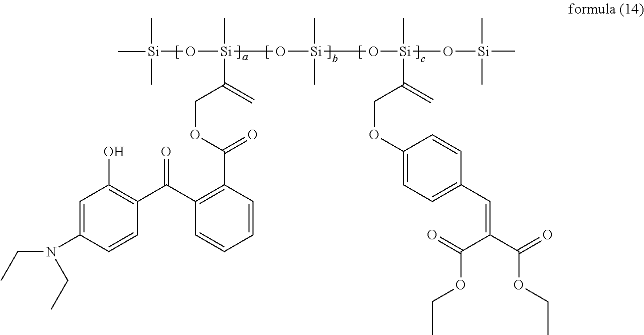 [SMILES: C=C(COC(=O)c1ccccc1C(=O)c1ccc(N(CC)CC)cc1O)[Si](C)(O[Si](C)(C)C)O[Si](C)(C)O[Si](C)(O[Si](C)(C)C)C(=C)COc1ccc(C=C(C(=O)OCC)C(=O)OCC)cc1]

Suitable organic UV-protecting substances can preferably be selected from the following list: Ethylhexyl salicylate, phenylbenzimidazolesulfonic acid, benzophenone-3, benzophenone-4, benzophenone-5, n-Hexyl 2-(4-diethylamino-2-hydroxybenzoyl)benzoate, 4-methylbenzylidenecamphor, terephthalylidenedicamphorsulfonic acid, disodium phenyldibenzimidazoletetrasulfonate, methylenebis(benzotriazolyl)tetramethylbutyl-phenol, Ethylhexyltriazone, Diethylhexylbutamidotriazone, Drometrizole trisiloxane, Polysilicone-15, 1,1-Dicarboxy(2,2′-dimethylpropyl)-4.4-diphenylbutadiene, 2,4-Bis[5-1 (dimethylpropyl)benzoxazol-2-yl(4-phenyl)imino]-6-(2-ethylhexyl)imino-1,3,5-triazine and mixtures thereof.

These organic UV filters are generally incorporated into formulations in an amount of 0.01 to 20% by weight, preferably 1 to 20% by weight.

Besides the extract and the optional organic UV filters, as described above, the preparations may comprise further inorganic UV filters, so-called particulate UV filters. These combinations with particulate UV filters are possible both as powder and also as dispersion or paste of the following types. Preference is given here both to those from the group of the titanium dioxides, such as, for example, coated titanium dioxide (for example Eusolex® T-2000, Eusolex® T-AQUA, Eusolex® T-AVO, Eusolex® T-OLEO), zinc oxides (for example Sachtotee), iron oxides or also cerium oxides and/or zirconium oxides. Furthermore, combinations with pigmentary titanium dioxide or zinc oxide are also possible, where the particle size of these pigments is greater than or equal to 200 nm, for example Hombitan® FG or Hombitan® FF-Pharma.

It may furthermore be preferred for the preparations to comprise inorganic UV filters which have been aftertreated by conventional methods, as described, for example, in Cosmetics & Toiletries 1990, 105, 53. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa;
Aftertreated micronised titanium dioxides with aluminium oxide and silicon dioxide aftertreatment, such as, for example, the product “Microtitanium Dioxide MT 100 SA from Tayca, or the product “Tioveil Fin” from Unidema;
Aftertreated micronised titanium dioxides with aluminium oxide and/or aluminium stearate/laurate aftertreatment, such as, for example, Micro-titanium Dioxide MT 100 T from Tayca; Eusolex T-2000 from Merck;
Aftertreated micronised titanium dioxides with iron oxide and/or iron stearate aftertreatment, such as, for example, the product “Microtitanium Dioxide MT 100 F” from Tayca; Aftertreated micronised titanium dioxides with silicon dioxide, aluminium oxide and silicone aftertreatment, such as, for example, the product “Microtitanium Dioxide MT 100 SAS”, from Tayca;
Aftertreated micronised titanium dioxides with sodium hexametaphosphate, such as, for example, the product “Microtitanium Dioxide MT 150 W” from Tayca.

The treated micronised titanium dioxides employed for the combination may also be aftertreated with:
Octyltrimethoxysilanes, such as, for example, the product Tego Sun T 805 from Degussa;
Silicon dioxide; such as, for example, the product Parsol T-X from DSM;
Aluminium oxide and stearic acid; such as, for example, the product UV-Titan M160 from Sachtleben;
Aluminium and glycerine; such as, for example, the product UV-Titan from Sachtleben,
Aluminium and silicone oils, such as, for example, the product UV-Titan M262 from Sachtleben;
Sodium hexamethaphosphate and polyvinylpyrrolidone,
Polydimethylsiloxanes, such as, for example, the product 70250 Cardre UF TiO2SI3″ from Cardre;
Polydimethylhydrogenosiloxanes, such as, for example, the product Microtitanium Dioxide USP Grade Hydrophobic” from Color Techniques.

The combination with the following products may furthermore also be advantageous:
Untreated zinc oxides, such as, for example, the product Z-Cote from BASF (Sunsmart), Nanox from Element is;
Aftertreated zinc oxides, such as, for example, the following products;
   “Zinc Oxide CS-5” from Toshibi (ZnO aftertreated with polymethylhydrogenosiloxane);
   Nanogard Zinc Oxide FN from Nanophase Technologies;
   “SPD-Z1” from Shin-Etsu (ZnO aftertreated with a silicone-grafted acrylic polymer, dispersed in cyclodimethylsiloxanes);
   “Escalol Z100” from ISP (aluminium oxide-aftertreated ZnO, dispersed in an ethylhexyl methoxycinnamate/PVP-hexa-decene/methicone copolymer mixture);
   “Fuji ZNO-SMS-10” from Fuji Pigment (ZnO aftertreated with silicon dioxide and polymethylsilesquioxane);
   Untreated cerium oxide micropigment, for example with the name “Colloidal Cerium Oxide” from Rhone Poulenc;
   Untreated and/or aftertreated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda. In addition, mixtures of aluminium oxide-, silicon dioxide- and silicone-aftertreated titanium dioxide/zinc oxide mixtures, such as, for example, the product UV-Titan M261 from Sachtleben, can also be employed.

These inorganic UV filters are generally incorporated into the preparations in an amount of 0.1 to 25% by weight, preferably 2 to 10% by weight.

By combination of one or more of the said compounds having a UV filter action, the reduction of the tanning effect of UV radiation can be optimised.

All said UV filters can also be employed in encapsulated form. In particular, it is advantageous to employ organic UV filters in encapsulated form. The capsules in preparations to be employed in accordance with the invention are preferably present in amounts which ensure that the encapsulated UV filters are present in the preparation in the per cent by weight ratios indicated above.

Further active compounds in the sense of the present invention are light-protection filters which absorb light in the wavelength range from 400 to 800 nm (VIS) and in the infrared wavelength region (IR) from 800 nm. The suitable choice of light-protection filters is important for the purposes of the present invention. Suitable light-protection filters absorb light from the said wavelength regions, but not the therapeutic wavelength, so that the therapeutic light is able to reach the skin at the site of action without being absorbed. Common light-protection filters are disclosed in EP 0898955 A2.

In accordance with the invention, light-protection filters can be pigments and/or dyes which reflect and/or absorb in the visible wavelength region (VIS-reflecting). Such pigments can be, in particular, golden, red, orange, copper or body-coloured interference pigments which come very close to the natural skin colour.

These interference pigments are preferably flake-form or ground mica having a diameter of up to 15 pm which is coated with SnO₂ and/or TiO₂. However, interference pigments whose support material does not consist of mica are also suitable. The coatings may be doped differently, such as, for example, by iron or cerium.

In a particular embodiment of these pigments, they are mica having a thin coating consisting of up to one % by weight of SnO₂ and a coating consisting of 50 to 70% by weight, preferably 54 to 60% by weight, of TiO₂ having a rutile structure.

The light-protection filters can also be mica having a thin coating consisting of up to one % by weight of SnO₂ and a coating consisting of 50 to 70% by weight, preferably 54 to 60% by weight, of TiO₂ having an anatase structure or mica having a coating consisting of 50 to 70% by weight, preferably 54 to 60% by weight, of TiO₂ having a rutile or anatase structure.

Suitable substances which can also be employed as VIS and/or as IR filters are pearlescent pigments consisting of mica or other support materials which have been coated with titanium dioxides or iron oxides. In particular, they are

silver pigments (mica+TiO₂) having particle sizes <200 μm, in particular <15 μm, such as, for example, the commercially available Timiron MP 1005 TM or MP 1001 TM or also coarser fractions interference pigments (mica+TiO₂) having particle sizes <200 μm, in particular having particle sizes of 5 to 25 μm, having golden, red, orange, copper or body-coloured interference, such as, for example, Timiron Silk Red™ or Silk Gold™ or Super Red™ or Super Gold™ or Super Copper™ or coarser fractions or other interference colours and mixtures thereof gold pigments (mica+TiO₂ and iron oxides) having particle sizes <200 μm, in particular <5 to 25 μm or <15 μm; a gold pigment of this type is, for example, Timiron MP 20™ , but also coarser gold-pigment fractions are suitable coloured pigments (mica+TiO₂ and iron oxides) having particle sizes <200 μm, in particular <5 to 25 μm or <15 μm; corresponding coloured pigments are, for example, Dichrona™ or Microna™ Matte.

Also suitable are VIS-absorbent or -reflective fillers, such as, for example, mica coated with TiO₂ and/or BaSO₄. These also include, for example, Biron™ (BiOCl), Low Luster™ or Extender W™, so long as they are not 100% transparent

Possible light-protection filters can also mixtures of the pigments, pearlescent pigments, VIS-reflective or absorbent fillers or dyes mentioned above and below as light-protection filters.

Normally employed as UV filters, microfine ZnO and TiO₂ particles are also suitable as such, so long as they also reflect or absorb in the VIS region. These are commercially available under the names Hombitec ™ or Sachotec™, Kemira M160™, Tioveil AQ™ and to a limited extent Eusolex T-2000™, to a limited extent since it has very high transparency.

Suitable VIS filters can also be dyes approved in cosmetics, for example selected from the “Blue List® (list of dyes approved in cosmetics) [”Blue List” Edition Cantor Verlag, ed. H. P. Fiedler (1993)], which CaO be employed both as such and also in a mixture. These dyes can also be employed as undissolved pigments. Particularly suitable here are the red, yellow and blue dyes, which, individually or in a mixture with the other additives, result in formulations which exhibit a natural coloration on application to the skin, Dyes from this list can therefore also be employed with colours other than those mentioned, such as, for example, orange or gold.

The red dyes employed are preferably those of the names D&C Red, preferably having the numbers No. 10, C.I. 15630, No. 7, C.I. 15850 and No. 21, C.I. 45380, Acid Red, preferably Acid Red No. 1, C.I. 18050, Allura Red, trans-alpha-, beta- or gamma-carotene, Pigment Red. The yellow dyes are those having the names Acid Yellow, preferably Acid Yellow No. 1, C.I. 10316, Tartrazine, C.I. 19140, Rutin, D&C Yellow No. 7, C.I. 45350, Disperse Yellow, Food Yellow, Natural Yellow, Pigment Yellow, Solvent Yellow.

Corresponding blue dyes are Acid Blue, preferably Acid Blue No. 9, C.I. 42090, Acid Blue No. 80, C.I. 61585, D&C Blue No. 6, C.I. 73000, C-Blue 21, Direct Blue 86.

Apart from the dyes listed in the said list, further VIS-absorbent substances are also suitable, such as, for example, flavonoids or natural or synthetic melanin.

Furthermore, the VIS filters may also have a protective action in the UV or IR region in addition to their protective action in the VIS region.

Protection of the skin against R radiation is likewise sensible and important, since the IR radiation of sunlight makes a significant contribution to warming. This heat is in turn synergistic in erythema formation caused by UV, i.e. it promotes sunburn formation.

Suitable IR-protection filters here are in principle many substances described for the VIS region, in particular the interference pigments which act in the longer-wave region. The transition between the VIS and the IR region is frequently fluid.

Pigments which reflect in the IR region are thus preferably employed. However, the strong “whitewashing” on the skin is often regarded as being in need of improvement. This problem is solved by the provision of a novel interference pigment having an action in the IR wavelength region.

This interference pigment consists of flake-form or ground mica which is coated with TiO₂ in different layer thicknesses and which may furthermore also be doped with iron or cerium.

The interference pigments have hues in the region of copper-coloured, yellowish and skin-coloured/pink. In order to describe the colours better, the hues can also be indicated in accordance with codes from the “Pantone Color Formula Guide 1000”, which are known to the person skilled in the art. The following hues are particularly preferred: 726 C, 489U, 4890, 7120, 155U, 719U, 1205U or also 12050. This list should merely be regarded as descriptive disclosure which is absolutely not limiting in any way.

The pigments exhibit a white mass tone, i.e. the formulations have a white colour, but then, as desired, a copper- or skin/pink-coloured interference colour appears on the skin. The undesired “whitewashing” does not occur here.

The interference pigments are prepared by the generally known methods for the continuous layer build-up of Ti(OH)₄ on mica particles (for example described in the documents U.S. Pat. No. 4,038,099, German Patent 25 22 572 or also EP 0 271 767 B1). The process is then stopped at the desired interference colour.

The particle size is of major importance for the effectiveness. The particle size is very particularly preferably from 5 to 25 μm, since an optimum protective action against IR radiation can thus be achieved.

If the particle size is selected smaller than approximately 15 μm, this interference pigment may then also be highly suitable for the VIS region.

The interference pigments for the VIS region can also be prepared, for example, by the processes described in the cited documents.

The filters for protection against VIS and IR radiation can in each case be incorporated into cosmetic formulations in concentrations of 0.5 to 20% by weight, preferably 3 to 10% by weight. These are substances which are dissolved, dispersed or emulsified with water and oils in a simple manner.

The light-protection filters can be incorporated directly into cosmetic formulations without further preparatory measures.”

Furthermore, the preparations according to the invention may comprise at least one further active compound. The further active compound is preferably selected from the group of UV filters, antioxidants, vitamins, skin-lightening active compounds, anti-ageing active compounds, anti-inflammatory active compounds, antimicrobial active compounds, active compounds for improving the moisture content of the skin (skin moisture regulators), anticellulite active compounds, antiwrinkle active compounds, antidandruff active compounds, anti-acne active compounds, deodorants, pigments and self-tanning substances, particularly preferably from the group of UV filters, antioxidants, vitamins, anti-ageing active compounds and anticellulite active compounds, very particularly preferably from the group of UV filters, antioxidants, vitamins and skin-lightening active compounds.

In a preferred embodiment of the invention, the preparation furthermore comprises at least one substance which serves for maintaining and/or improving the moisture content of the skin. These substances can, without this being intended to be regarded as a restriction, also be, inter alia, substances which belong to the so-called natural moisturising factors. in a further preferred embodiment of the invention, the preparation comprises one or more antioxidants and/or one or more vitamins. The use of antioxidants enables a protective action against oxidative stress or against the effect of free radicals in general to be achieved, the person skilled in the art being presented with absolutely no difficulties in selecting antioxidants which act suitably quickly or with a time delay. There are many proven substances known from the specialist literature which can be used as antioxidants, for example amino acids (for example glycine, histidine, tyrosine, tryptophan) and derivatives thereof, and derivatives thereof, peptides, such as, for example, D,L-carnosine, D-carnosine, L-carnosine and derivatives thereof (for example anserine), carotinoids, carotenes (such as, for example, α-carotene, β-carotene, lycopene) and derivatives thereof, chlorogenic acid and derivatives thereof aurothioglucose, propylthio-uracil and other thiols (such as, for example, thioredoxin, glutathione, cysteine, cystine, cystamine and the glycosyl, N-acetyl, methyl, ethyl, propyl, amyl, butyl and lauryl, palmitoyl, oleyl, cholesteryl and glyceryl esters thereof) and salts thereof, dilauryl thiodipropionate, distearyl thiodipropionate, thiodipropionic acid and derivatives thereof (such as, for example, esters, ethers, peptides, lipids, nucleotides, nucleosides and salts), and sulfoximine compounds (such as, for example, buthionine sulfoximines, homocysta sulfoximine, buthionine sulfones, penta-, hexa- and heptathionine sulfoximine) in very low tolerated doses (such as, for example, pmol to μmol/kg), and also (metal) chelating agents, (such as, for example, α-hydroxyfatty acids, palmitic acid, phytic acid, lactoferrin), α-hydroxy acids (such as, for example, citric acid, lactic acid, malic acid), humic acid, bile acid, bile extracts, bilirubin, biliverdin, EDTA, pentasodium ethylenediamine tetramethylene phosphonate and derivatives thereof, unsaturated fatty acids and derivatives thereof, vitamin C and derivatives (such as, for example, ascorbyl palmitate, magnesium ascorbyl phosphate, ascorbyl acetate), tocopherols and derivatives (such as, for example, vitamin E acetate), vitamin A and derivatives (such as, for example, vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (such as, for example, ZnO, ZnSO₄). Further suitable antioxidants are also described in WO 2006/111233 and WO 2006/111234.

Suitable antioxidants are also compounds of the general formulae (15) or (16)

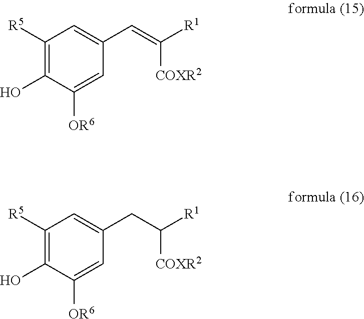

in which
R¹ denotes —C(O)CH₃, —CO₂R³, —C(O)NH₂ and —C(O)N(R⁴)₂,
X denotes 0 or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁶ denotes H. linear or branched alkyl having 1 to 8 C atoms or linear or branched alkoxy having 1 to 8 C atoms, and
R⁶ denotes linear or branched alkyl having 1 to 8 C atoms.

Preference is given to derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl)2-(4-hydroxy-3,5-dimethoxybenzy)malonate (for example RonaCare® AP).

Mixtures of antioxidants are likewise suitable for use in the preparations according to the invention. Known and commercial mixtures are, for example, mixtures comprising, as active ingredients, lecithin. L-(+)-ascorbyl palmitate and citric acid, natural tocopherols. L-(+)-ascorbyl palmitate, L-(+)-ascorbic acid and citric acid (such as, for example, Oxynex® K LIQUID), tocopherol extracts from natural sources. L-(+)-ascorby) palmitate. L-(+)-ascorbic acid and citric acid (such as, for example, Oxynex® L LIQUID), DL-a-tocopherol, L-(+)-ascorbyl palmitate, citric acid and lecithin (such as, for exam O ple, xynex® LM) or butylhydroxytoluene (BHT). L-(+)ascorbyl palmitate and citric acid (such as, for example, Oxynex®2004). Antioxidants of this type are usually employed in such compositions with compounds of the formula (I) or part-formulae thereof in per cent by weight ratios in the range from 1000:1 to 1:1000, preferably in per cent by weight ratios of 100:1 to 1:100.

Of the phenols having an antioxidative action, the polyphenols, some of which are naturally occurring, are of particular interest for applications in the pharmaceutical, cosmetic or nutrition sector. For example, the flavonaids or bioflavonoids, which are principally known as plant dyes, frequently have an antioxidant potential. Lemanska et al., Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example Rice-Evans et al., Trends in Plant Science 1997, 2(4), 152-159). Lemanska et al., Free Radical Biology & Medicine 2001, 31(7), 869-881, have investigated the pH dependence of the antioxidant action of hydroxyflavones. Quercetin exhibits the highest activity amongst the structures investigated over the entire pH range.

The preparations according to the invention may comprise vitamins as further ingredients. Vitamins and vitamin derivatives selected from vitamin A, vitamin A propionate, vitamin A palmitate, vitamin A acetate, retinal, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K1, esculin (vitamin P active compound), thiamine (vitamin B₁), nicotinic acid (niacin), pyridoxine, pyridoxal, pyridoxamine, (vitamin B₆), panthothenic acid, biotin, folic acid and cobalamine (vitamin B₁₂) are preferably present in the preparations according to the invention, particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the preparations in ranges from 0.01 to 5% by weight, based on the total weight.

The preparations according to the invention may in addition comprise anti-ageing active compounds, anticellulite active compounds or conventional skin-protecting or skin-care active compounds. Skin-protecting or skin-care active compounds can in principle be all active compounds known to the person skilled in the art. Particularly preferred anti-ageing active compounds are pyrimidinecarboxylic acids, aryl oximes, bioflavonoids, bio-flavonoid-containing extracts, chromones or retinoids.

Suitable anti-ageing active compounds, in particular for skin-care preparations, are preferably also so-called compatible solutes. These are substances which are involved in the osmoregulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycero-phosphorylcholines, glutamine, glycine, α-alanine, glutamate, aspartate, proline, and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidine-carboxylic acids (such as ectoin and hydroxyectoin), proline, betaine, glutamine, cyclic diphosphoglycerate, N-acetylornithine, trimethylamine N-oxide, di-myo-inositol phosphate (DIP), cyclic 2,3-diphosphoglycerate (cDPG), 1,1-diglycerol phosphate (DGP), p-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidine-carboxylic acid) and derivatives thereof.

Known anti-ageing substances are also chromones, as described, for example, in EP 1508327, or retinoids, for example retinal (vitamin A), retinoic acid, retinaldehyde or also synthetically modified compounds of vitamin A. The chromones and retinoids described are simultaneously also effective anticellulite active compounds. An anticellulite active compound which is likewise known is caffeine.

The following, for example, may be mentioned as use form of the preparations according to the invention: solutions, suspensions, emulsions. PIT emulsions, pastes, ointments, gels, creams, lotions, powders, soaps, surfactant-containing cleansing preparations, oils, aerosols, plasters, compresses, bandages and sprays, in particular for external application. Further application forms are, for example, sticks, shampoos and shower baths. Typcal cosmetic use forms are furthermore also lipsticks, lip-care sticks, powder, emulsion and wax make-up, and sun-protection, pre-sun and after-sun preparations.

Cosmetic and dermatological preparations according to the invention can be, in particular, a water-free preparation, a lotion or emulsion, such as cream or milk, or microemulsion, in each case of the water-in-oil (W/O ) type or of the oil-in-water (O/W) type, a multiple emulsion, for example of the water-in-oil-in-water (W/O/W) type or vice versa (O/W/O), gels or solutions (in particular oily-alcoholic, oily-aqueous or aqueous-alcoholic gels or solutions), a solid stick, an ointment or an aerosol. For application, the cosmetic and dermatological preparations according to the invention are applied to the skin in adequate amount in the usual manner for cosmetics.

An embodiment of the invention is an emulsion which is in the form of a cream or milk and comprises, for example, fatty alcohols, fatty acids, fatty acid esters, in particular triglycerides of fatty acids, lanolin, natural and synthetic oils or waxes and emulsifiers in the presence of water. Further particularly preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids. A particularly preferred preparation according to the invention may also be in the form of an alcoholic gel which comprises one or more lower alcohols or polyols, such as ethanol, propylene glycol or glycerol, and a thickener, such as siliceous earth. The oily-alcoholic gels additionally comprise natural or synthetic oil or wax. The solid sticks preferably consist of natural or synthetic waxes and oils, fatty alcohols, fatty acids, fatty acid esters, lanolin and other fatty substances. If a preparation is formulated as an aerosol, the usual propellants, such as alkanes, air, nitrogen, dinitrogen monoxide, particularly preferably alkanes or air, are preferably used.

Any desired conventional vehicles, assistants and, if desired, further active compounds may be added to the preparation. Preferred assistants originate from the group of the preservatives, stabilisers, solubilisers, colorants, i.e. pigments, dyes, emulsifiers or odour improvers.

The present invention accordingly also relates to preparations, characterised in that a vehicle which is suit able for cosmetic, pharmaceutical or and/or dermatological applications and optionally physiologically acceptable assistants and/or fillers are present.

Suitable vehicles and assistants or fillers are described in detail in the following part.

Ointments, pastes, creams and gels may comprise the customary vehicles which are suitable for topical application, such as, for example, animal and vegetable fats, waxes, paraffins, starch, tragacanth, cellulose derivatives, polyethylene glycols, silicones, bentonites, silica, talc and titanium dioxide, or mixtures of these substances.

Powders and sprays may comprise the customary vehicles, such as, for example, lactose, talc, silica, aluminium hydroxide, calcium silicate and polyamide powder, or mixtures of these substances. Sprays may additionally comprise the customary readily volatile, liquefied propellants, such as, for example, chlorofluorocarbons, propane/butane or dimethyl ether. Compressed air can also advantageously be used. However, air can also be employed in pressureless metering devices, such as, for example, pump sprays. Solutions and emulsions may comprise the customary vehicles, such as solvents, solubilisers and emulsifiers, such as, for example, water, ethanol, isopropanol, ethyl carbonate, ethyl acetate, benzyl alcohol, benzyl enzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexane-carbonyl-D-alanine methyl ester.

Suspensions may comprise the customary vehicles, such as liquid diluents, such as, for example, water, ethanol or propylene glycol, suspension media, such as, for example, ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances. Soaps may comprise the customary vehicles, such as alkali-metal salts of fatty acids, salts of fatty acid mono-esters, fatty acid protein hydrolysates, isothionates, lanolin, fatty alcohol, vegetable oils, plant extracts, glycerol, sugars, or mixtures of these substances. Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances. Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

The preferred preparation forms according to the invention include, in particular, emulsions. O/W emulsions are particularly preferred. Emulsions. W/O emulsions and O/W emulsions can be obtained in a conventional manner. Emulsions according to the invention are advantageous and comprise, for example, the said fats, oils, waxes and other fatty substances, as well as water or an aqueous phase, for example with solvents or hydrophilic surfactants, and an emulsifier, as usually used for a preparation of this type.

The lipid phase can advantageously be selected from the following substance group:
mineral oils, mineral waxes;
oils, such as, for example, triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes, and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group of the esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n butyl stearate, n-hexyl laurate, n-decyl oleate, isooctyl stearate, isononyl stearate, isononyl isononanoate, 2-ethylhexyl palmitate, 2-ethylhexyl laurate, 2-hexaldecyl stearate, 2-octyldodecyl palmitate, oleyl oleate, oleyl erucate, erucyl oleate, erucyl erucate and synthetic, semi-synthetic and natural mixtures of esters of this type, such as, for example, jojoba oil. Furthermore, the oil phase can advantageously be selected from the group of branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides can advantageously be selected, for example, from the group of synthetic, semi-synthetic and natural oils, for example olive oil, sunflower oil, soya oil, peanut oil, rapeseed oil, almond oil, palm oil, coconut oil, palm kernel oil and the like. Any desired mixtures of oil and wax components of this type may also advantageously be employed for the purposes of the present invention. It may also be advantageous to employ waxes, for example cetyl palmitate, as sole lipid component of the oil phase.

The aqueous phase of the preparations according to the invention optionally advantageously comprises alcohols, diols or polyols having a low carbon number, and ethers thereof, preferably ethanol, isopropanol, propylene glycol, glycerol, ethylene glycol, ethylene glycol monoethyl or mono-butyl ether, propylene glycol monomethyl, monoethyl or monobutyl ether, diethylene glycol monomethyl or monoethyl ether and analogous products, furthermore alcohols having a low carbon number, such as, for example, ethanol, isopropanol, 1,2-propanediol, glycerol, and, in particular, one or more thickeners, which may advantageously be selected from the group of silicon dioxide, aluminium silicates, polysaccharides or derivatives thereof, such as, for example, hyaluronic acid, xanthan gum, hydroxypropylmethyl-cellulose, particularly advantageously from the group of the polyacrylates, preferably a polyacrylate from the group of the so-called Carbopols, for example Carbopol grades 980, 981, 1382, 2984, 5984, in each case individually or in combination. In particular, mixtures of the above-mentioned solvents are used. In the case of alcoholic solvents, water may be a further constituent.

In a preferred embodiment, the preparations according to the invention comprise hydrophilic surfactants. The hydrophilic surfactants are preferably selected from the group of the alkylglucosides, acyl lactylates, betaines and coconut amphoacetates.

Emulsifiers that can be used are, for example, the known W/O and O/W emulsifiers. It is advantageous to use further conventional co-emulsifiers in the preferred O/W emulsions according to the invention.

The co-emulsifiers selected in accordance with the invention are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols).

It is furthermore advantageous to select the fatty acid ethoxylates from the following group: polyethylene glycol (20) stearate, polyethylene glycol (21) stearate, poly-ethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth1-4 sulfate. An ethoxylated cholesterol derivative which can advantageously be used is poly-ethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group of polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, poly-ethylene glycol (20) glyceryl isostearate, polyethylene glycol (18) glyceryl oleate (cocoate.

It is likewise favourable to select the sorbitan esters from the group poly-ethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

The following can be employed as optional W/O emulsifiers, but ones which may nevertheless be advantageous in accordance with the invention: fatty alcohols having 8 to 30 carbon atoms, monoglycerol esters of saturated and/or unsaturated, branched and/or unbranched alkanecarboxylic acids having a chain length of 8 to 24, in particular 12 to 18 C atoms, diglycerol esters of saturated and/or unsaturated, branched and/or unbranched alkanecarboxylic acids having a chain length of 8 to 24, in particular 12 to 18 C atoms, monoglycerol ethers of saturated and/or unsaturated, branched and/or unbranched alcohols having a chain length of 8 to 24, in particular 12 to 18 C atoms, diglycerol ethers of saturated and/or unsaturated, branched and/or unbranched alcohols having a chain length of 8 to 24, in particular 12 to 18 C atoms, propylene glycol esters of saturated and/or unsaturated, branched and/or unbranched alkanecarboxylic acids having a chain length of 8 to 24, in particular 12 to 18 C atoms, and sorbitan esters of saturated and/or unsaturated, branched and/or unbranched alkanecarboxylic acids having a chain length of 8 to 24, in particular 12 to 18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol mono-caprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments, which colour the composition itself or the skin, and other ingredients usually used in cosmetics.

The dispersant or solubiliser used can be an oil, wax or other fatty substances, a lower monoalcohol or a lower polyol or mixtures thereof. Particularly preferred monoalcohols or polyols include ethanol, i-propanol, propylene glycol, glycerol and sorbitol.

The preparations according to the invention can be prepared with the aid of techniques which are well known to the person skilled in the art. The mixing can result in dissolution, emulsification or dispersion of the active compounds in the vehicle.

In order to increase the protective or therapeutic action of the compounds according to the invention, pharmaceutically tolerated adjuvants can be added. For the purposes of the invention, any substance which facilitates, enhances or modifies an effect with the skin-lightening compounds in accordance with the invention is an “adjuvant”. Known adjuvants are, for example, aluminium compounds, such as, for example, aluminium hydroxide or aluminium phosphate, saponins, such as, for example, QS 21, muramyl dipeptide or muramyl tripeptide, proteins, such as, for example, gamma-interferon or TNF, ME 59, phosphatdibylcholine, squalene or polyols. Furthermore, DNA which encodes a protein with an adjuvant effect can be applied in parallel or in a construct.

The introduction of the pharmaceutical composition into a cell or organism can be carried out in accordance with the invention in any manner which enables the melanin concentration to be reduced. The pharmaceutical composition of the present invention can be administered orally, topically, transdermally, transmucosally, transurethrally, vaginally, rectally, pulmonarily, enterally and/or parenterally, preferably topically or transdermally. The type of administration selected depends on the indication, the dose to be administered, individual-specific parameters, etc. In particular, the various types of administration facilitate site-specific therapy, which minimises side effects and reduces the active-compound dose.

The administration forms of the pharmaceutical composition are prepared corresponding to the desired type of administration in a suitable dosage and in a manner known per se using the customary solid or liquid vehicles and/or diluents and the assistants usually employed. Thus, pharmaceutically acceptable excipients known to the person skilled in the art can basically form part of the pharmaceutical composition according to the invention, where the amount of excipient material which is combined with the active compound in order to prepare a single dose varies depending on the individual to be treated and the type of administration. These pharmaceutically tolerated additives include salts, buffers, fillers, stabilisers, complexing agents, antioxidants, solvents, binders, lubricants, tablet coatings, flavours, dyes, preservatives, adjusters and the like. Examples of excipients of this type are water, vegetable oils, benzyl alcohols, alkylene glycol, polyethylene glycol, glycerol triacetate, gelatine, carbohydrates, such as, for example, lactose or starch, magnesium stearate, talc and Vaseline.

The pharmaceutical formulation can be in the form of a tablet, film tablet, dragee, lozenge, capsule, pill, powder, granules, syrup, juice, drops, solution, dispersion, suspension, suppository, emulsion, implant, cream, gel, ointment, paste, lotion, serum, oil, spray, aerosol, adhesive, plaster or bandage. Oral administration forms which are prepared are preferably tablets, film tablets, dragees, lozenges, capsules, pills, powders, granules, syrups, juices, drops, solutions, dispersions or suspensions—including as depot form. Furthermore, parenteral medicament forms, such as, for example, suppositories, suspensions, emulsions, implants or solutions, should be considered, preferably oily or aqueous solutions. For topical application, the medicament active compound is formulated in a conventional manner with at least one pharmaceutically acceptable vehicle, such as, for example, microcrystalline cellulose, and optionally further assistants, such as, for example, moisturisers, to give solid formulations which can be applied to the skin, such as, for example, creams, gels, ointments, pastes, powders or emulsions, or to give liquid formulations which can be applied to the skin, such as, for example, solutions, suspensions, lotions, sera, oils, sprays or aerosols. The pharmaceutical composition is preferably in a form for topical application. The pharmaceutical composition may also be in the form of a solid composition, for example in the lyophilised state, and can then be prepared before use by addition of a dissolving agent, such as, for example, distilled water. The person skilled in the art is familiar with the basic principles of the preparation of lyophilisates.

A “prophylactic effect” prevents the outbreak of a disease and also includes an increase in normal physiological function. Prophylaxis is advisable, in particular, if an individual has predispositions for the onset of the above-mentioned diseases, such as, for example, a family history, a gene defect or a recently survived disease. A “therapeutically relevant effect” results in part or full freedom from one, more than one or all disease symptoms or results in the partial or complete return of one, more than one or all physiological or biochemical parameters which are associated with or causally involved in the disease or pathological change to the normal state. The respective dose or dose range for the administration is sufficiently large to achieve the desired prophylactic or therapeutic effect of induction of a biological or medical response. In general, the dose will vary with the age, constitution and gender of the patient, and the severity of the disease will be taken into account. It goes without saying that the specific dose, frequency and duration of administration are, in addition, dependent on a multiplicity of factors, such as, for example, the targeting and bonding ability of the compounds, feeding habits of the individual to be treated, type of administration, excretion rate and combination with other drugs. The individual dose can be adjusted both with respect to the primary disease and also with respect to the occurrence of any complications. The precise dose can be established by a person skilled in the art using known means and methods.

In order to support the medicinal effect, the pharmaceutical composition may, in an embodiment of the invention, also comprise one or more further active compounds, where simultaneous or successive administration is conceivable.

The present invention still furthermore relates to a kit (of parts) consisting of a said cosmetic or pharmaceutical or dermatological composition according to the invention and a device containing an artificial radiation source which is selected from the group consisting of laser, lamp, light-emitting diode (LED); organic light-emitting diode (OLED), polymeric light-emitting diode (PLED) and organic light-emitting electrochemical cell (OLEC. LED or also LEEC), preferably of lamp, LED, OLED, PLED and OLEC, very preferably of LED, OLED, PLED and OLEC and very particularly preferably of OLED, PLED and OLEC.

Finally, the present invention also relates to a method for the cosmetic and/or therapeutic treatment of the skin, characterised in that the skin is treated by the combined use of at least one skin-lightening compound and by irradiation by means of an artificial radiation source.

The present invention furthermore also relates to a method for the cosmetic and/or therapeutic treatment of the skin, characterised in that the skin is treated by the combined use of a combination of at least one light-protection filter and at least one skin-lightening compound and by irradiation by means of an artificial radiation source.

For the purposes of the present invention, the combined use of compounds and radiation is not automatically taken to mean the simultaneous use of the two components. The use can be carried out simultaneously, in an overlapping manner or successively.

The individual terms here have the meaning indicated above.

The treatment method may be characterised in that the treatment of the skin with the skin-lightening compound and the irradiation is carried out successively, in an overlapping manner or simultaneously. The skin, or parts thereof, can, for example, be treated and/or pretreated with the compositions described above at regular intervals. The skin must typically be pretreated in order to keep the melanin concentration during phototherapy low.

Preferred is also a method according to claim 14, characterised in that the treatment of the skin with the compound and the light source is carried out in an overlapping manner and preferably simultaneously.

In the method indicated, the radiation employed may be continuous or pulsed.

For the purposes of the present invention, the radiation sources indicated above are preferred.

For the purposes of the present invention, preference is furthermore given to the use of the wavelengths or wavelength ranges indicated above.

In a furthermore preferred embodiment of the present invention, the method for the pharmaceutical and/or cosmetic applications described in greater detail above is used. Particular preference is given here to the treatment of jaundice, very particularly preferably jaundice of the newborn and especially preferably jaundice in newborn having skin type III to VI.

The use according to the invention of skin-lightening compound in combination with phototherapy for the treatment of the skin, in particular for the treatment of jaundice of the newborn, is distinguished over the prior art by the surprising advantages mentioned above and below:
1. Better efficacy of the phototherapeutic radiation due to greater penetration of this radiation into the skin.
2. Fewer side effects due to lower phototherapeutic radiation doses which have to be employed owing to the greater efficacy.
3. Additional increase in efficacy of the phototherapeutic radiation and reduction of side effects due to the additional use of light-protection filters (UV, VIS, IR).
4. Reduction of long-term damage of the skin by phototherapy (for example reduction in the formation of liver spots).
5. Reduction of induced oxidative stress triggered by visible light.
6. Reduction in the drying-out of the skin which is observed in photo-therapeutic applications.

Even without further comments, it is assumed that a person skilled in the art will be able to utilise the above description in the broadest scope. It should be pointed out that variations of the embodiments described in the present invention fall within the scope of this invention. Each feature disclosed in the present invention can, unless explicitly excluded, be replaced by alternative features which serve the same, an equivalent or a similar purpose. Each feature disclosed in the present invention, unless stated otherwise, should thus be regarded as an example of a generic series or as an equivalent or similar feature.

All features of the present invention can be combined with one another in any way, unless certain features and/or steps are mutually exclusive. This applies, in particular, to preferred features of the present invention. Equally, features of non-essential combinations can be used separately (and not in combination).

It should furthermore be pointed out that many of the features, and in particular those of the preferred embodiments of the present invention, are themselves inventive and should not be regarded merely as part of the embodiments of the present invention. Independent protection for these features may be sought in addition or as an alternative to each invention claimed at present.

The teaching regarding technical action that is disclosed with the present invention can be abstracted and combined with other examples.

The invention is explained in greater detail by the following examples without wishing to restrict it thereby.

### EXAMPLES

### Example 1

Production of an OLEC having Blue Emission

The following polymers ILA and BE1 are prepared with the aid of Suzuki coupling. The process is well known to the person skilled in the art and is disclosed, for example, in WO 2003/048225.

Polymer IL1, which is used as interlayer, is a copolymer containing the two following monomers with the mol% indicated in each case:

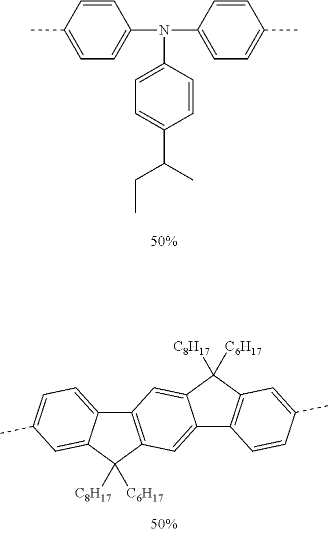

The molecular weight (MW) of polymer IL1 is distributed between 200000 to 300000 g/mol.

BE1 is a copolymer containing 50% of phenanthrene and 50% of spirobi-fluorene units. Copolymer BE1 exhibits a broad emission band between 400 and 500 nm.

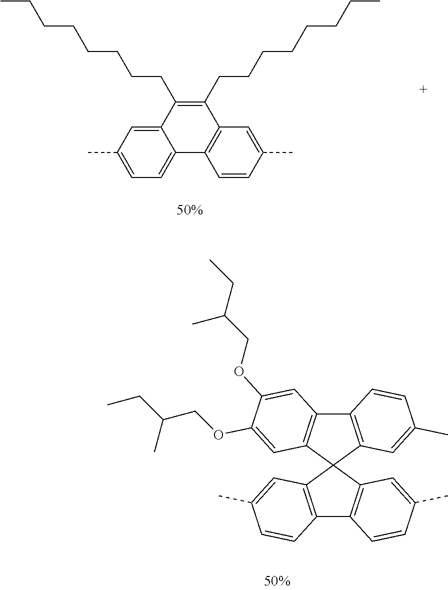

The molecular weight (MW) of polymer BE1 is distributed between 200000 to 300000 g/mol.

For the emission layer, use is made of a formulation Fl which is prepared as follows:
a solution A is prepared by dissolving BE1 in chloroform which comprises BE1 in a concentration of 10 mg/ml;
a solution B is prepared by dissolving PEO (polyethylene oxide as ion conductor having a molecular weight of MW=5×10⁶, Aldrich) and IM2 (tetrabutylammonium tetracyanoborate, Z. Anorg. Allg. Chem. 2000, 626, 560-568.) in a weight ratio of 20:1 in cyclohexane having a concentration of 20 mg/ml.

Formulation 1 is obtained by mixing solutions A and B in the specific ratio, so that the weight ratio of BE1:PEO:IM2 is equal to 1:1:0.2.

OLEC1 having the layer sequence Al cathode/EML/interlayer/PEDOT:PSS/ITO anode/substrate, where the emission layer (EML) comprises BE1, can be produced as follows.
1.) PEDOT (Baytron P Al 4083) is applied to the substrate in a thickness of 80 nm by means of spin coating and heated at 120° C. for 10 min. The substrate used is flexible polyethylene naphthalate (PEN); 150 nm of ITO are sprayed onto PEN;
2.) 20 nm of IL1 are applied in a glove box by spin coating from a toluene solution having a concentration of 0.5% by weight;
3.) the emission layer EML (having a thickness of 300 nm) is applied by spin coating of formulation 1; the application is carried out in a glove box;
4.) the device is heated at 120° C. for 30 min. in order to remove solvents remaining;
5.) an Al cathode (150 nm) is applied to the emission layer by evaporation;
6.) the device is encapsulated by means of UV-cured resin (UV Resin T-470/UR7114, Nagase Chemtex Corporation) in a glove box using methods which are well known to the person skilled in the art.

OLEC1 emits principally light in the wavelength range between 400 and 500 nm. The use of filters or colour converters enables the emission to be set to specific wavelengths depending on the application. In order to achieve emission at 466 nm, a filter is employed.

With BE1 as component in the EML, it is also possible to produce a PLED by methods which are well known to the person skilled in the art from the prior art.

### Example 2

Measurement of the Transmission of Brilirubin-Degrading Light of 466 nm by Various Melanocyte Cell Cultures

Procedure description B16 V mouse melanoma culture

B16V mouse melanoma cells (manufacturer: DSMZ; Article No.: ACC370) in RPMI medium (Invitrogen, Article No.: 31870), to which 10% of FBS (Invitrogen, Article No.: 10499044), 2 mM L-glutamine (Invitrogen, Article No: 25030) and 1 mM sodium pyruvate (Invitrogen, Article No.: 11360) had additionally been added, are transferred and incubated at 37° C. and 5% CO₂ for 72h. The medium is separated off, and the cells are washed once with 10 ml of DPBS (Invitrogen, Article No.: 14190) and subsequently 20 removed by suction. 1 ml of HyQtase cell detachment solution (Hyclone, Article No.: SV30030.01) is added to the cells. The bottle is swirled a number of times, and the HyQtase cell detachment solution is subsequently removed by suction. The cells are then incubated in the incubator at 37° C. and 5% CO₂ for 5 min. The cells are taken up in the modified RPMI medium (see above), and the cell count is determined. To this end, the cells are stained with Trypan Blue and counted in a Neubauer chamber. The cells are subsequently sown out again in the modified RPMI medium (see above) in a defined cell count of 80,000 cells per well (6-well clear plate, TCT, PS (Nunc)).

Incubation is subsequently carried out at 37° C. and 5% CO₂ for 24h, the medium is then removed. 1980 pl of the substance dilution is subsequently added. For this substance dilution, Eugenia uniflora extract is dissolved in DMSO and subsequently filtered through a sterile filter (0.2 μm, Millipore, Article No. SLLG013a). The solution is then diluted with the modified RPMI medium (in this case the FBS content in the RPMI medium is only 5%) in such a way that concentration of the extract is 0.1 mg/ml. 20 μl of an alpha-MSH (melanocyte-stimulating hormone) solution (DMSO, Sigma, Article No.:D2650) are then added, so that the alpha-MSH concentration in the well is 10⁻⁸ M. The plate is subsequently incubated again at 37° C. and 5% CO₂ for 24h. The process described in this section is repeated a further twice in total.

After the final incubation period, the medium is removed by suction, and the cells are washed with 1000 μl of DPBS (lnvitrogen, Article No.: 14190). Removal by suction is again carried out 250 μl of HyQtase cell detachment solution (Hyclone, Article No.: SV30030.01) are added to the cells. The 6-well plate is swirled a number of times, and the HyQtase cell detachment solution is subsequently removed by suction. The cells are then incubated in the incubator at 37° C. and 5% CO₂ for 5 min. The cells are then taken up in 1.5ml of DPBS (Invitrogen, Article No.: 14190) and transferred into cup (SARSTEDT. Ref. 72.692.005).

0.75 ml thereof were in each case returned in order subsequently to measure the transmission of blue light (samples A). The cell count is sub-sequently determined. To this end, the cells are stained with Trypan Blue and counted in a Neubauer chamber. The cells centrifuged for 1 min at 3500 g. The pellets obtained are photographed, and the supernatant is subsequently removed by suction. The pellets are dissolved in 1 ml of 1 N NaOH at 80° C. for 1h and then cooled to room temperature. 200 μl as quadruple determination (per cup) are then pipetted into a 96-well plate (VWR, Article No.: 4100636981), and the absorption at a wavelength of 405 nm is determined (Safire, Tecan). The content of melanin can be determined in this way by means of a calibration line.

As comparison, a sample without extract, but with 0.1% of DMSO and a sample without extract, but with 0.1% of DMSO and alpha-MSH (concentration in the well at 10⁻⁸ M) is used in parallel in each case.

The melanin content in the melanocytes is reduced by a Eugenia uniflora water extract.

Standardised to the sample comprising 0.1% of DMSO addition to 100%, the following representation arises

Percent of the respective control [μg/ml]

| | Percent [%] |
|---|---|
| DMSO (0.1%) | 100.0 |
| DMSO (0.1%) + α-MSH | 204.2 |
| Eugenia uniflora 0.1 mg/ml + α-MSH | 26.4 |

DMSO (0.1%)+α-MSH can also be formed in the skin during the treatment by phototherapy and corresponds to accelerated pigmentation of the newborn skin in the experiment.

DMSO (0.1%) corresponds to the normally pigmenting newborn skin without light stimulation.

Eugenia uniflora 0.1 mg/ml+α-MSH corresponds to the newborn skin with treatment of a melanin synthesis inhibitor and phototherapy.

The transmissivity for light of wavelength 466 nm is now measured in order to determine the efficiency of phototherapy in jaundice of the newborn. To this end, samples A are transferred into a cell, and the transmission at 466 nm is determined. The samples are irradiated using the electroluminescent device from Example 1. However, analogous results can also be achieved with other light sources (for example PLED, OLED). The sample corresponding to the untreated, unirradiated newborn skin (DMSO (0.1%) was set to 100%. The following transmission values arise:

% transmission at 466 nm

| | Percent [%] |
|---|---|
| DMSO (0.1%) | 100.0 |
| DMSO (0.1%) + α-MSH | 50 |
| Eugenia uniflora 0.1 mg/ml + α-MSH | 210 |

The therapy with melanin synthesis inhibitors with simultaneous photo-therapy with 466 nm in the case of jaundice of the newborn causes a significant increase in the efficiency of the treatment by achieving higher availability of the relevant radiation.

The experiment was repeated with the following skin-lightening compounds kojic acid, liquorice extract, emblica extract, arbutin, magnesium ascorbyl phosphate. L-ascorbic acid, ascorbyl glucosamine and azelaic acid. All cell cultures treated in this way exhibit increased transmission in the range between 180 and 245%.

### Example 3

Compositions of Skin-Lightening Compounds

O/W emulsions for retardation of melanin synthesis and skin care in newborn, data in % by weight

| Ingredients | 1-1 | 1-2 | 1-3 | 1-4 | 1-5 |
|---|---|---|---|---|---|
| Rucinol | 0.5 | 0.25 | 0.1 | 0.25 | 0.1 |
| Stearyl alcohol (and) | 3 | 3 | 3 | 3 | 3 |
| steareth-7 (and) steareth- | | | | | |
| 10 | | | | | |
| Glyceryl stearate (and) | 3 | 3 | 3 | 3 | 3 |
| ceteth-20 | | | | | |
| Glyceryl stearate | 3 | 3 | 3 | 3 | 3 |
| Microwax | 1 | 1 | 1 | 1 | 1 |
| Cetearyl octanoate | 11.5 | 11.5 | 11.5 | 11.5 | 11.5 |
| Caprylic/capric | 6 | 6 | 6 | 6 | 6 |
| triglyceride | | | | | |
| Oleyl oleate | 6 | 6 | 6 | 6 | 6 |
| Propylene glycol | 4 | 4 | 4 | 4 | 4 |
| Propylparaben | 0.05 | 0.05 | 0.05 | 0.05 | 0.05 |
| Methylparaben | 0.15 | 0.15 | 0.15 | 0.15 | 0.15 |
| Tromethamine | | | 1.8 | | |
| Water | To 100 | To 100 | To 100 | To 100 | To 100 |

### Example 4

Composition of Skin-Lightening Compounds Comprising Light-Protection Filters (UV)—W/O Emulsion

| | 54-07-5-A | 54-07-5-B | 54-07-5-C | 54-07-5-D | 54-07-5-E |
|---|---|---|---|---|---|
| Cetyl PEG/PPG-10/1 | 3.00 | 3.00 | 3.00 | 3.00 | 3.00 |
| dimethicone | | | | | |
| (Abil EM 90) | | | | | |
| Polyglyceryl-4 | 1.50 | 1.50 | 1.50 | 1.50 | 1.50 |
| isostearate | | | | | |
| (Isolan GI 34) | | | | | |
| Butylphthalimide | 5.00 | 5.00 | 5.00 | 5.00 | 5.00 |
| isopropylphthalimide | | | | | |
| (Pelemol ® BIP) | | | | | |
| Dimethyl isosorbide | 5.00 | 5.00 | 5.00 | 5.00 | 5.00 |
| (Arlasolve DMI) | | | | | |
| (R)-2-((R)--3,4- | 1.00 | | | 1.00 | 2.00 |
| Dihydroxy-5-oxo-2,5- | | | | | |
| dihydrofuran-2-yl)-2- | | | | | |
| hydroxyethyl 2-(4- | | | | | |
| dihexylylamino-2- | | | | | |
| hydroxybenzoyl)- | | | | | |
| benzoate | | | | | |
| Uvinul ® A Plus (DHHB) | | 0.84 | 0.84 | 1.00 | |
| Ascorbic acid | | | 0.37 | 1.00 | 3.00 |
| Rucinol | 0.5 | 1 | 2 | 3 | 5 |
| Mineral oil | 8.00 | 8.00 | 8.00 | 8.00 | 8.00 |
| Ethylhexyl stearate | 5.00 | 5.00 | 5.00 | 5.00 | 5.00 |
| (Tegosoft ® OS) | | | | | |
| Cyclomethicone (and) | 5.00 | 5.00 | 5.00 | 5.00 | 5.00 |
| Aluminium/Magnesium | | | | | |
| Hydroxide Stearate | | | | | |
| (Gilugel SIL 5) | | | | | |
| Preservative | 1.00 | 1.00 | 1.00 | 1.00 | 1.00 |
| Water | To 100 | To 100 | To 100 | To 100 | To 100 |
| NaCl | 0.50 | 0.50 | 0.50 | 0.50 | 0.50 |
| EDTA | 0.10 | 0.10 | 0.10 | 0.10 | 0.10 |
| Citric acid q.s. | | | | | |

Preparation: Pelemol® BIP, Arlasolv DMI and emulsifiers are initially introduced. (R)-2-((R)--3,4-Dihydroxy-5-oxo-2,5-dihydrofuran-2-yl)-2-hydroxy-ethyl 2-(4-dihexylylamino-2-hydroxybenzoyl)benzoate and Uvinul® A Plus are dissolved therein. The remaining constituents of the oil phase are added and mixed homogeneously. The water phase, adjusted to pH=4-5, is emulsified in with stirring. The mixture is subsequently homogenised. The emulsions can be prepared under gentle conditions at room temperature. By increasing the content of ascorbic acid, (R)-2-((R)--3,4-dihydroxy-5-oxo-2,5-dihydrofuran-2-yl)-2-hydroxyethyl 2-(4-dihexylyl-amino-2-hydroxybenzoyl)benzoate can be stabilised. The preparation is ideally under an inert atmosphere (exclusion of oxygen prepared).

### Example 5

Composition of Skin-Lightening Compound Comprising Additional Active Compound

| Ingredients | 1-1 | 1-2 | 1-3 | 1-4 | 1-5 |
|---|---|---|---|---|---|
| Emblica TM (Merck) | 0.5 | 0.25 | 0.1 | 0.25 | 0.1 |
| RonaCare Tiliroside | 1 | 0.5 | 0.3 | 0.1 | 0.05 |
| RonaCare Ectoin | 6 | 2 | 1 | 0.5 | 0.1 |
| Stearyl alcohol (and) | 3 | 3 | 3 | 3 | 3 |
| steareth-7 (and) steareth- | | | | | |
| 10 | | | | | |
| Glyceryl stearate (and) | 3 | 3 | 3 | 3 | 3 |
| ceteth-20 | | | | | |
| Glyceryl stearate | 3 | 3 | 3 | 3 | 3 |
| Microwax | 1 | 1 | 1 | 1 | 1 |
| Cetearyl octanoate | 11.5 | 11.5 | 11.5 | 11.5 | 11.5 |
| Caprylic/capric | 6 | 6 | 6 | 6 | 6 |
| triglyceride | | | | | |
| Oleyl oleate | 6 | 6 | 6 | 6 | 6 |
| Propylene glycol | 4 | 4 | 4 | 4 | 4 |
| Propylparaben | 0.05 | 0.05 | 0.05 | 0.05 | 0.05 |
| Methylparaben | 0.15 | 0.15 | 0.15 | 0.15 | 0.15 |
| Tromethamine | | | 1.8 | | |
| Water | To 100 | To 100 | To 100 | To 100 | To 100 |

1.-19. (canceled)

20. A skin-lightening compound for the therapeutic treatment of the skin with simultaneous use of phototherapy, wherein an artificial radiation source is used for the phototherapy.

21. The compound according to claim 20, wherein jaundice is treated with the compound.

22. Compound according to claim 20, wherein psoriasis, atopic dermatitis eczema of the skin, inflammation of the skin, reddening of the skin and/or acne is treated.

23. mpound according to claim 20, wherein the artificial radiation source is selected from the group consisting of laser, lamp, light-enlifting diode (LED); organic light-emitting diode (OLED), polymeric light-emitting diode (PLED) and organic light-emitting electrochemical cell (OLEC, LEC or also LEEC).

24. The compound according to claim 20, wherein the radiation source emits continuous or pulsed radiation.

25. The compound according to claim 20, wherein the compound is selected from the group consisting of hydroquinone, kojic acid, liquorice extract, arbutin, azelaic acid, alpha lipoic acid, rucinol, vitamin C and derivatives, niacinamide, and aloesin.

26. The compound according to claim 20, wherein the radiation source emits radiation having a wavelength and/or having a wavelength range between 250 and 2000 nm.

27. A method comprising utilizing a skin-lightening compound in cosmetic phototherapeutic applications.

28. A pharmaceutical composition comprising at least one compound according to claim 20 and optionally at least one further pharmaceutical assistant.

29. The pharmaceutical composition according to claim 28, comprising at least one further active compound.

30. A cosmetic composition comprising at least one compound according to claim 20 and optionally at least one further cosmetic assistant.

31. The cosmetic composition according to claim 30, comprising at least one further active compound.

32. A kit consisting of the composition according to claim 28 and a device containing an artificial radiation selected from the group consisting of laser, lamp, light-emitting diode (LED); organic light-emitting diode (OLED), polymeric light-emitting diode (PLED) and organic light-emitting electrochemical cell (OLEC, LEC or also LEEC).

33. A method for the cosmetic and/or therapeutic treatment of the skin, comprising treating the skin with a combination of at least one skin-lightening compound and irradiation by means of an artificial radiation source.

34. The method according to claim 33, wherein treating the skin with the compound and the light source is carried out in an overlapping manner and/or simultaneously.

35. The method according to claim 33, wherein the radiation source emits continuous radiation or pulsed radiation.

36. The method according to claim 33, wherein the radiation source is selected from the group consisting of laser, lamp, light-emitting diode (LED); organic light-emitting diode (OLED), polymeric light-emitting diode (PLED) and organic light-emitting electrochemical cell (OLEC. LEC or also LEEC).

37. The method according to claim 33, wherein the radiation source emits radiation having a wavelength and/or having a wavelength range between 250 and 2000 nm.

38. The method according to claim 33, wherein jaundice is treated.
